(19) United States
(12) Patent Application Publication
(10) Pub. No.: US 2009/0325244 A1
(43) Pub. Date: Dec. 31, 2009

(54) METHOD OF INCREASING GENE EXPRESSION USING MODIFIED CODON USAGE

(75) Inventors: Andrea Herold, Ketsch (DE); Corinna Klopprogge, Mannheim (DE); Hartwig Schröder, Nussloch (DE); Oskar Zelder, Speyer (DE); Weol Kyu Jeong, Gunsan (KR)

Correspondence Address: CONNOLLY BOVE LODGE & HUTZ, LLP, P O BOX 2207, WILMINGTON, DE, 19899, US

(73) Assignee: BASF SE, LUDWIGSHAFEN (DE)

(21) Appl. No.: 12/446,809

(22) PCT Filed: Oct. 18, 2007
(86) PCT No.: PCT/EP07/61152

§ 371 Date: Jun. 10, 2009

(30) Foreign Application Priority Data
Oct. 24, 2006 (EP) 06122868.0

(51) Int. Cl. C12P 13/12; C12N 15/87; C12N 15/82; C12N 15/11; C12N 15/00; C12N 1/21; C12P 13/04; C12P 13/08; C12P 7/64

(52) U.S. Cl. 435/113; 435/455; 435/468; 435/471; 536/23.1; 536/23.7; 435/320.1; 435/252.32; 435/106; 435/115; 435/134

(57) ABSTRACT

The present invention relates to a method of increasing the amount of at least one polypeptide in the host cell by expressing a modified nucleotide sequence encoding for a polypeptide in a host cell with said modified nucleotide sequence being derived from a different non-modified nucleotide sequence encoding for a polypeptide of identical amino acid sequence such that the codon usage of the modified nucleotide sequence is adjusted to the codon usage of abundant proteins in the host cell.

### OBJECT OF THE INVENTION

The present invention relates to a method of increasing the amount of at least one polypeptide in a host cell wherein the codon usage of the nucleotide sequence which is to be expressed is adjusted to the codon usage of abundant proteins of the host cell.

The present invention also relates to nucleotide sequences encoding for a polypeptide with a codon usage that has been adjusted to the codon usage of abundant proteins in the host cell. Such nucleotide sequences allow for increased expression of the respective polypeptide.

The present invention is also concerned with a method of increasing the amount of at least one polypeptide in Corynebacterium glutamicam wherein the codon usage of the nucleotide sequence which is to be expressed is adjusted to the codon usage of Corynebacterium glutamicum.

The present invention also relates to nucleotide sequences encoding for a polypeptide with a codon usage that has been adjusted to the codon usage of Corynebacterium glutamicum. Such nucleotide sequences allow for increased expression of the respective polypeptide.

The present invention further relates to the use of the aforementioned nucleotide sequences for overexpressing the polypeptides encoded thereby to increase production of fine chemicals.

### BACKGROUND

In a lot of biotechnological processes it is necessary to modulate gene expression. Thus, for some applications it is necessary to increase the expression of a certain gene product and to thereby increase the amount and/or activity of e.g. a protein in the host cell in which the gene of Interest is (over)expressed. Similarly, it may be desirable to reduce the amount of expression of an endogenous gene in a host cell.

The fermentative production of so-called fine chemicals is today typically carried out in microorganisms such as Corynebacterium glutamicum (C. glutamicum), Escherichia coli (E. coli), Saccharomyces cerevisiae (S. cerevisiae), Schizzosaccharomyes pombe (S. pombe), Pichia pastoris (P. pastoris), Aspergillus niger, Bacillus subtilis, Ashbya gossypii or Gluconobacter oxydans.

Fine chemicals which include e.g. organic acids such as lactic acid, proteogenic or non-proteogenic amino acids, purine and pyrimidine bases, carbohydrates, aromatic compounds, vitamins and cofactors, lipids, saturated and unsaturated fatty acids are typically used and needed in the pharmaceutical, agriculture, cosmetic as well as food and feed industry.

As regards for example the amino acid methionine, currently worldwide annual production amounts to about 500,000 tons. The current industrial production process is not by fermentation but a multi-step chemical process. Methionine is the first limiting amino acid in livestock of poultry feed and due to this mainly applied as a feed supplement. Various attempts have been published in the prior art to produce methionine e.g. using microorganisms such as E. coli.

Other amino acids such as glutamate, lysine, threonine and threonine, are produced by e.g. fermentation methods. For these purposes, certain microorganisms such as C. glutamicum have proven to be particularly suited. The production of amino acids by fermentation has the particular advantage that only L-amino acids are produced and that environmentally problematic chemicals such as solvents as they are typically used in chemical synthesis are avoided.

A lot of the attempts in the prior art to produce fine chemicals such as amino acids, lipids, vitamins or carbohydrates in microorganisms such as E. coli and C. glutamicum have attempted to achieve this goal by e.g. increasing the expression of genes involved in the biosynthetic pathways of the respective fine chemicals. If e.g. a certain step in the biosynthetic pathway of an amino acid such as methionine or lysine is known to be rate-limiting, over-expression of the respective enzyme may allow obtaining a microorganism that yields more product of the catalysed reaction and therefore will ultimately lead to an enhanced production of the respective amino acid.

Attempts to increase production of e.g. methionine and lysine by upregulating the expression of genes being involved in the biosynthetic pathway of methionine or lysine production are e.g. described in WO 02/10209, WO 2006008097, WO2005059093 or in Cremer et al. (Appl. Environ. Microbiol, (1991), 57(6), 1746-1752).

Typically, overexpression of a certain gene in a microorganism such as E. coli or C. glutamicum or other host cells such as P. pastoris, A. niger or even mammalian cell culture systems may be achieved by transforming the respective cell with a vector that comprises a nucleotide sequence encoding for the desired protein and which further comprises elements that allow the vector to drive expression of the nucleotide sequence encoding e.g. for a certain enzyme. Using this approach foreign proteins, i.e. proteins that are encoded by sequences that are not naturally found in the host cell that is used for expression, as well as endogenous host cell-specific proteins may be overexpressed. Other typical methods include increasing the copy number of the respective genes in the chromosome, inserting strong promoters for regulating the transcription of the chromosomal copy of the respective genes and enhancing translational initiation by optimization of the ribosomal binding site (RBS).

The expression of foreign genes in a certain host cell may be particularly desirable as this approach allows to confer novel and unique characteristics to a host cell if e.g. a gene encoding for a certain enzymatic activity is introduced which naturally is not found in the host cell.

However, overexpression of foreign genes having no counterpart in the host cell by using e.g. expression vectors such as plasmids has encountered problems. The same has been observed for overexpression of genes which have a counterpart, in the host organism as regards their function but which use a nucleotide sequence that is typically not found within the host organism. The failure of host cells such as E. coli or C. glutamicum to express certain foreign (heterologous) sequences may be due to altered codon usage (see e.g. WO 2004042059).

The genetic code is degenerate which means that a certain amino acid may be encoded by a number of different base triplets. Codon usage refers to the observation that a certain organism will typically not use every possible codon for a certain amino acid with the same frequency. Instead an organism will typically show certain preferences, i.e. a bias for specific codons meaning that these codons are found more frequently in the transcribed genes of an organism. One explanation for different codon usages in different organisms may be that the genes encoding for the respective tRNA and tRNA isoacceptors differ in the degree to which they are expressed and thus available during translation.

Organism-specific codon usage can be one of the reasons why e.g. translation of a synthetic-gene or a foreign gene even when coupled to a strong promoter often proceeds much more slowly than would be accepted. This lower than expected translation efficiency is explained by that the protein's coding regions of the gene have a codon usage pattern that does not resemble that of the host cells.

As codon usage is highly biased and varies considerably in different organisms, introduction of a foreign sequence that has a different codon usage bias than the host organism can alter a peptide elongation rates as the host organism will have to produce e.g. more of the respective tRNAs.

There are different codon-optimisation techniques available for improving, the translational kinetics of translationally inefficient protein coding regions. These techniques mainly rely on identifying the codon usage for a certain host organism. If a certain gene or sequence should be expressed in this organism, the coding sequence of such genes and sequences wilt then be modified such that one will replace codons of the sequence of interest by more frequently used codons of the host organism.

However, even for known codon optimisation approaches, there remain efficiency problems as regards the expression of e.g. foreign genes.

In view of this situation, it is one object of the present invention to provide codon usage data for industrially important microorganisms such as C. glutamicum on the basis of which improved expression of coding sequences can be achieved. Furthermore, it is an object of the present invention to provide new methods for codon optimisation which allow circumvention of the drawbacks of the prior art.

These and other objectives as they will become apparent from the ensuing description of the invention are solved by the present invention as described in the independent claims. The dependent claims relate to preferred embodiments.

### SUMMARY OF THE INVENTION

In one aspect the invention is concerned with a method of increasing the amount of at least one polypeptide in a host cell. The method comprises the step of expressing a polypeptide-encoding sequence which has been adjusted to the codon usage of abundant proteins of the host organism.

in particular, the method comprises the step of expressing a modified nucleotide sequence which encodes for said at least one polypeptide in said host cell. The modified nucleotide sequence is derived from a different starting nucleotide sequence such that the codon usage of the modified nucleotide sequence is adjusted to the codon usage of abundant proteins of the respective host organism. The modified nucleotide sequence and the starting nucleotide sequence encode for substantially the same amino acid sequence and/or function. Both sequences usually encode for identical amino acid sequences at least at the positions where adjustment to the codon usage of abundant proteins has been introduced.

Modification of the starting nucleotide sequence will usually be done by replacing at least one codon of the starting nucleotide sequence by a codon that is more frequently used in the group of abundant polypeptides of the host organism.

As regards increasing the expression of polypeptides on the basis of the codon usage of abundant host proteins, at least one, at least two, at least three, at least four, at least five, at least six, at least seven, at least eight, at least, nine, at least ten, preferably at least 1%, at least 2%, at least 4%, at least 6%, at least 8%, at least 10%, more preferably at least 20%, at least 40%, at least 60%, at least 80%, even more preferably at least 90% or least 95% and most preferably all codons of the starting nucleotide sequence are replaced in the modified nucleotide sequence by more frequently and preferably by the most frequently used codons for the respective amino acid as determined for the group of abundant proteins. In a particularly preferred embodiment the afore-mentioned number of codons to be replaced refers to rare, very rare and particularly extremely rare codons. In an even more preferred embodiment these codons are replaced by frequent, very frequent, extremely frequent or the most frequent codons. In another particularly preferred embodiment, the number of codons to be replaced refers to the least frequently used codons which are replaced by the most frequently used codons.

In one embodiment of the invention, the method will make use of modified nucleotide sequences which use for each amino acid the most frequently used codon of the abundant proteins of the respective host cell.

The at least one polypeptide that is expressed according to the above described method may be a polypeptide originating from organisms different than said host cell, i.e. a foreign polypeptide, or it may be a polypeptide of said host cell, i.e. an endogenous polypeptide with the proviso that the modified nucleotide sequence is different from the starting sequence encoding a polypeptide of substantially the same amino acid and/or function.

Host cells may be selected from microorganisms including bacteria and fungi, insect cells, plant cells or mammalian cell culture systems.

Using the inventive method, it is possible to overexpress a polypeptide in a host cell. Thus, using the inventive method the amount of the expressed polypeptide may be increased by at least 5%, 10%, 20%, 30%, 40%, 50%, 60%, 70%, 80%, 90% or 100%. In preferred embodiments the amount of the polypeptide may be increased by a factor of a least 3, 4, 5, 6, 7, 8, 9 or 10 or even more preferably by a factor of at least 20, 50, 100, 500 or 1,000. The increased amount of expressed polypeptide refers to a comparison of expression of the modified nucleotide sequence with expression of the starting nucleotide sequence under comparable conditions (e.g. same host cell, same vector type etc.).

In a preferred embodiment, a method in accordance with the invention relates to increasing the amount of at least one polypeptide in the genus Corynebacterium. A particularly preferred embodiment relates to increasing the amount in C. glutamicum.

These preferred embodiments of the invention comprise the step of expressing a modified nucleotide sequence encoding for a polypeptide. The modified nucleotide sequence is derived from a different starting nucleotide sequence such that the codon usage of the modified nucleotide sequence is adjusted to the codon usage of the group of abundant proteins in the genus of Corynebacterium and particularly preferably of C. glutamicum. Both the modified and the starting nucleotide sequence will encode for substantially the same amino acid sequence and/or function. Both sequences usually encode for identical amino acid sequences at least at the positions where the modifications have been introduced.

As set out above, this preferred embodiment of the invention may be used to overexpress endogenous or foreign polypeptides. The method may also be used to overexpress mutants of certain proteins. For example, the method may be used to overexpress certain mutant enzymes which have been desensitized as regards feed back inhibition compared to the wild type enzymes.

As regards the expression of polypeptides in the genus of Corynebacterium and particularly in the species of C. glutamicum by modified codon usage, at least one, at least two, at least three, at least four, at least five, at least six, at least seven, at least eight, at least nine, at least ten, preferably at least 1%, at least 2%, at least 4%, at least 6%, at least 8%, at least 10%, more preferably at least 20%, at least 40%, at least 60%, at least 80%, even more preferably at least 90% or least 95% and most preferably all codons of the starting nucleotide sequences are replaced in the modified nucleotide sequence by more frequently and preferably by the most frequently used codons for the respective amino acid as determined for the group of abundant proteins. In a particularly preferred embodiment, the afore-mentioned number of codons to be replaced refers to rare, very rare and particularly extremely rare codons. In an even more preferred embodiment these codons are replaced by frequent, very frequent, extremely frequent or the most frequent codons. In another particularly preferred embodiment, the number of codons to be replaced refers to the least frequently used codons which are replaced by the most frequently used codons.

For expression of polypeptides in the genus of Corynebacterium and particularly in the species of C. glutamicum by optimised codon usage, in another embodiment the starting nucleotide sequence encoding for the polypeptide may be modified such that at least one, at least two, at least three, at least four, at least five, at least six, at least seven, at least eight, at least nine, at least ten, preferably at least 1%, at least 2%, at least 4%, at least 6%, at least 8%, at least 10%, more preferably at least 20%, at least 40%, at least 60%, at least 80%, even more preferably at least 90% or least 95% and most preferably all codons of the starting nucleotide sequence are replaced in the resulting modified nucleotide sequence by more frequently and preferably by the most frequently used codons for the respective amino acid according to Table 2. In particularly preferred embodiment the afore-mentioned number of codons to be replaced refers to rare, very rare and particularly extremely rare codons. In another particularly preferred embodiment, the number of codons to be replaced refers to the least frequently used codons which are replaced by the most frequently used codons of Table 2.

Further preferred embodiments of the invention relate to methods of increasing the amount of a polypeptide in Corynebacterium and particularly preferably in C. glutamicum wherein at least one, at least two, at least three, at least four, at least five, at least six, at least seven, at least eight, at least nine, at least ten, preferably at least 1%, at least 2%, at least 4%, at least 6%, at least 8%, at least 10%, more preferably at least 20%, at least 40%, at least 60%, at least 80%, even more preferably at least 90% or least 95% and most preferably all codons of the starting nucleotide sequence are replaced in the resulting modified nucleotide sequence by one of the two most frequently used codons for the respective amino acid according to Table 2. In an even more preferred embodiment the afore-mentioned number of codons to be replaced refers to rare, very rare and particularly extremely rare or the least frequently used codons.

In a particularly preferred embodiment of the invention, the method will rely on modified nucleotide sequences using the codons GUU for valine, GCU for alanine, GAC for aspartic acid, GAG for glutamic acid and/or ATG for methionine if ATG is the start codon.

In yet another embodiment of the invention which relates to a method of increasing the amount of polypeptide in Corynebacterium and particularly preferably in C. glutamicum, at least one codon of the aforementioned modified nucleotide sequences will be selected from Table 3.

Another particularly preferred embodiment of the invention relates to methods of increasing the amount of a polypeptide in Corynebacterium and particularly preferably in C. glutamicum wherein at least one, at least two, at least three, at least four, at least five, at least six, at least seven, at least eight, at least nine, at least ten, preferably at least 1%, at least 2%, at least 4%, at least 6%, at least 8%, at least, 10%, more preferably at least 20%, at least 40%, at least 60%, at least 80%, even more preferably at least 90% or least 95% and most preferably all codons of the starting nucleotide sequence are replaced in the resulting modified nucleotide sequence by the codons for the respective amino acid according to Table 3. In an even more preferred embodiment the afore-mentioned number of codons to be replaced refers to rare, very rare and particularly extremely rare codons.

Another particularly preferred embodiment of the invention relate to methods of increasing the amount of a polypeptide in Corynebacterium and particularly preferred in C. glutamicum wherein all codons of the starting nucleotide sequence are replaced in the resulting modified nucleotide sequence by the codons for the respective amino acid according to Table 3.

As regards the embodiments of the invention that relate to increasing the amount of a polypeptide in the host organism Corynebacterium and particularly preferably in C. glutamicum by using codon optimisation that is based on the codon usage of abundant proteins in Corynebacterium and particularly preferably in C. glutamicum, the methods may be used to overexpress the at least one polypeptide by the same amounts as has been set out above in general. Again, the increase in amount of polypeptide obtained by expression following a method in accordance with the invention is determined in comparison to expression of the starting original sequence in Corynebacterium and particularly preferably in C. glutamicum.

In some of the preferred embodiments of the invention, the above described method of increasing the amount of a polypeptide in host cells and preferably in Corynebacterium and particularly preferably in C. glutamicum may be used for producing fine chemicals such as amino acids, sugars, lipids, oils, carbohydrates, vitamins, cofactors etc.

For these purposes the modified nucleotide sequences may be selected from sequences encoding genes of biosynthetic pathways that are involved in the production of the aforementioned fine chemicals and for which overexpression is known to enhance production of the line chemical(s).

In one particularly preferred embodiment, methods in accordance with the invention may thus be used to produce fine chemicals such as amino acids and particularly amino acids such as lysine, threonine, cysteine and methionine.

Yet another embodiment of the present invention relates to the modified nucleotide sequences which are used for expression of a polypeptide in a host cell that have been derived from the different, starting nucleotide sequences encoding for polypeptides of substantially the same amino acid sequence and/or function by adjusting the codon usage of the modified nucleotide sequences to the codon usage of the group of abundant proteins of the respective host cell.

Of course, the invention in a preferred embodiment also relates to such modified nucleotide sequences that have been derived for a specific polypeptide, be it of foreign or endogenous origin with or without additional mutations, by replacing at least one, at least two, at least three, at least four, at least five, at least six, at least seven, at least eight, at least nine, at least ten, preferably at least 1%, at least 2%, at least 4%, at least 6%, at least 8%, at least 10%, more preferably at least 20%, at least 40%, at least 60%, at least 80%, even more preferably at least 90% or least 95% and most preferably all codons of the starting nucleotide sequences in the modified nucleotide sequence by more frequently and preferably by the most frequently used codons for the respective amino acid as determined for the group of abundant, proteins of the respective host organism. In an even more preferred embodiment the afore-mentioned numbers of codons to be replaced refer to rare, very rare and particularly extremely rare codons.

In one preferred embodiment of the invention, the modified nucleotide sequence uses for each amino acid the most frequently used codon of the abundant proteins of the respective host cell.

In case of modified nucleotide sequences that are to be (over)expressed in Corynebacterium and particularly preferably in C. glutamicum at least one, at least two, at least three, at least four, at least five, at least six, at least seven, at least eight, at least nine, at least ten, preferably at least 1%, at least 2%, at least 4%, at least 6%, at least 8%, at least 10%, more preferably at least 20%, at least 40%, at least 60%, at least 80%, even more preferably at least 90% or least 95% and most preferably all of the codons of the starting nucleotide sequences are replaced in the modified nucleotide sequence by more frequently and preferably by the most frequently used codons for the respective amino acid as determined for the group of abundant proteins of C. glutamicum. In an even more preferred embodiment the afore-mentioned numbers of codons to be replaced refer to rare, very rare and particularly extremely rare codons.

In case of modified nucleotide sequences that are to be (over)expressed in Corynebacterium and particularly preferably in C. glutamicum, at least one, at least two, at least three, at least four, at least five, at least six, at least seven, at least eight, at least nine, at least ten, preferably at least 1%, at least 2%, at least 4%, at least 6%, at least 8%, at least 10%, more preferably at least 20%, at least 40%, at least 60%, at least 80%, even more preferably at least 90% or least 95% and most preferably all codons of the starting nucleotide sequence are replaced in the resulting modified nucleotide sequence by more frequently used codons for the respective amino acid according to Table 2. In an even more preferred embodiment the afore-mentioned numbers of codons to be replaced refer to rare, very rare and particularly extremely rare codons.

For other preferred embodiments as far as nucleotide sequences for increasing expression in Corynebacterium and particularly preferably in C. glutamicum is concerned, at least one, at least two, at least three, at least four, at least five, at least six, at least seven, at least eight, at least nine, at least ten, preferably at least 1%, at least 2%, at least 4%, at least 6%, at least 8%, at least 10%, more preferably at least 20%, at least 40%, at least 60%, at least 80%, even more preferably at least 90% or least 95% and most preferably all codons of the starting nucleotide sequence are replaced in the resulting modified nucleotide sequence by one of the two most frequently used codons for the respective amino acid according to Table 2. In an even more preferred embodiment the afore-mentioned number of codons to be replaced refers to rare, very rare and particularly extremely rare codons.

As regards a particularly preferred embodiment of the modified nucleotide sequences, codons of the modified nucleotide sequence will use GUU for valine, GCU for alanine, GAG for aspartic acid, GAG for glutamic acid and/or ATG for methionine if ATG is the start codon.

The modified nucleotide sequence which is used for expression of the polypeptide in Corynebacterium and particularly preferably in C. glutamicum may also use codons that are selected from the codon usage of Table 3.

Thus, another particularly preferred embodiment of the invention relates to modified nucleotide sequences for increasing the amount of a polypeptide in Corynebacterium and particularly preferred in C. glutamicum wherein at least one, at least two, at least three, at least four, at least five, at least six, at least seven, at least eight, at least nine, at least ten, preferably at least 1%, at least 2%, at least 4%, at least 6%, at least 8%, at least 10%, more preferably at least 20%, at least 40%, at least 60%, at least 80%, even more preferably at least 90% or least 95% and most preferably all codons of the starting nucleotide sequence are replaced in the resulting modified nucleotide sequence by the codons for the respective amino acid according to Table 3. In an even more preferred embodiment the afore-mentioned numbers of codons to be replaced refer to rare, very rare and particularly extremely rare codons.

Another particularly preferred embodiment of the invention relates to modified nucleotide sequences for increasing the amount of a polypeptide in Corynebacterium and particularly preferably in C. glutamicum wherein all codons of the starting nucleotide sequence are replaced in the resulting modified nucleotide sequence by die codons for the respective amino acid according to Table 3.

Other embodiments of the invention relate to vectors that comprise the aforementioned nucleotide sequences and which are suitable for expression of a polypeptide in a host cell.

Yet another embodiment of the present invention relates to host cells which comprise the aforementioned modified nucleotide sequences or the aforementioned vectors.

The present invention also relates to the use of the aforementioned methods, nucleotide sequences, vectors and/or host cells for producing line chemicals such as amino acids, lipids, oils, carbohydrates, vitamins, cofactors etc.

The aforementioned methods, nucleotide sequences, vectors and host cells may particularly be used for production of fine chemicals such as amino acids including lysine, threonine, cysteine, and methionine.

The above described methods for increasing the amount of a polypeptide in a host cell rely on an optimisation of codon usage on the basis of the codon frequency of abundant proteins of the respective host cell. Optimisation means that when designing the modified nucleotide sequence preferably such codons are avoided which have been found to be rarely used in the group of abundant proteins of the respective host cells. Instead such codons are selected that are more (and preferably most) frequently used for the specific amino acid according to the codon usage of abundant proteins of the host cell.

However, the present invention not only relates to codon optimisation as described above, but in one embodiment also to preserving the distribution frequency of codon usage in the original starting sequence and the modified sequence. For example, instead of replacing a rarely used codon in the original starting sequence with a more frequently used host-specific codon, one may substitute the codon of the starting sequence with a codon of the host cell that is used at a comparable frequency in abundant proteins of the host cell. As far as Corynebacterium and C. glutamicum in particular is concerned, one may rely in this context also on the data of Table 2 from which one can infer the distribution frequency of codons in abundant proteins.

Of course, the present invention also relates to nucleotide sequences in which the distribution frequency of codon usage is adjusted to the distribution frequency of codon usage of abundant proteins. Similarly, vectors and host cells comprising such nucleotide sequences form part of the invention as well as the use of such methods, nucleotide sequences, vectors and host cells for producing fine chemicals.

Yet another embodiment of the present invention relates to methods for increasing the amount of polypeptide in Corynebacterium and particularly preferably in C. glutamicum in which a modified nucleotide sequence is expressed wherein the sequence of the modified nucleotide sequence has been adjusted to the codon usage of the complete organism C. glutamicum as set forth in Table 1. In methods relating to this aspect of the invention a modified nucleotide sequence may be used wherein at least one, at least two, at least three, at least four, at least five, at least six, at least seven, at least eight, at least nine, at least ten, preferably at least 1%, at least 2%, at least 4%, at least 6%, at least 8%, at least 10%, more preferably at least 20%, at least 40%, at least 60%, at least 80%, even more preferably at, least 90% or least 95% and most preferably all of the rare codons of the starting nucleotide sequence are replaced in the resulting modified nucleotide sequence by more frequently and preferably the most frequently used codons for the respective amino acid according to Table 1. In an even more preferred embodiment the afore-mentioned numbers of codons to be replaced refer to rare, very rare and particularly extremely rare codons.

In another preferred embodiment of this latter aspect of the invention, a modified nucleotide sequence may be used wherein all codons of the starting nucleotide sequence are replaced in the resulting modified nucleotide sequence by more frequently and preferably the most frequently used codons for the respective amino acid according to Table 1.

The present invention also relates to the nucleotide sequences which have been optimised on the basis of the codon usage of the organism C. glutamicum. Cloning and expression vectors and host cells which comprise these sequences also form part of the invention. The present invention relates as well to the use of such sequences for producing fine chemicals such as those mentioned above.

### FIGURES

FIG. 1 a) shows the codon usage optimised sequence of Lysine-23-aminomutase of Clostridium subterminale (SEQ ID No. 1). FIG. 1 b) shows the complete insert which has been cloned into pClik 5a MCS (p Clik 5a MCS Fsod Synth Kam A, SEQ ID No 2.). Underlined are the SpeI-recognition sites. The pSOD promoter is in italics. The codon usage optimised sequence of Lysine-2,3-aminomutase is in bold and the terminator sequence is grey shadowed.

FIG. 2 shows the expression constructs that were used for expressing the non-modified sequence and codon usage optimised Lysine-2,3-aminomutase of C. subterminale.

FIG. 3 shows an SDS-PAGE gel picture in which expression of codon usage optimised versus non-modified Lysine-2,3-aminomutase expression of C. subterminale was determined In C. glutamicum. Lanes M represent a pre-stained protein standard (SeeBlue Prestained Standard, Invitrogen). Lanes 1,2 represent expression from pClik 5a MCS. Lanes 3,4 represent expression from pClik 5a MCS Psod synth. KamA (FIG. 2a). Lanes 5,6 represent expression from pClik 5a MCS genomisch KamA Cl sub (FIG. 2b).

FIG. 4 shows the expression construct that was used for expressing the non-modified sequence of Lysine-2,3-aminomutase of C. subterminale under the control of the Psod promoter.

FIG. 5 shows an SDS-PAGE gel picture in which expression of codon usage optimised versus non-modified lysA was determined in C. glutamicum. Lane 1 represents a pre-stained protein standard (SeeBlue Prestained Standard. Invitrogen), Lanes 2,3 represent expression from pClik 5a MCS. Lanes 4,5 represent” expression from pClik 5a MCS genomisch KamA Cl sub (FIG. 2b). Lanes 6,7 represent expression from pClik 5a MCS Psod synth. KamA (FIG. 2a). Lanes 8,9 represent expression from pClik 5a MCS genom KamA (FIG. 4). The arrow indicates lysA.

FIG. 6 shows the codon usage optimised sequence of diaminopimelate decarboxylase (lysA (SEQ ID No. 8).

FIG. 7 shows the codon usage optimised sequence of lysA including up- and downstream regions. The restriction sites are underlined and the coding sequence is in bold. The upstream and downstream sequences are in italics (SEQ ID No. 9).

FIG. 8 shows the expression construct of codon usage optimised lysA for expression in C, glutamicum.

FIG. 9 shows an SDS-PAGE gel picture in which expression of codon usage optimised versus non-modified metH was determined in C. glutamicum. Lanes 1-3 represent expression of optimised metH, Lane 4 represents expression from empty vector. Lane 5 represents expression of wild type metH. The arrow indicates metH.

### DETAILED DESCRIPTION OF THE INVENTION

In one aspect, the present invention relies partly on the surprising finding that, determination of the codon usage of an organism may give different results depending on whether the codon usage is determined only for abundant proteins or for the organism as a whole.

Typically, codon usage tables in the prior art for organisms such as E. coli etc. have been based on an analysis of the complete genome. The inventors of the present invention have surprisingly found for the case of C. glutamicum that codon usage analysis of abundant proteins will give quite different results compared to codon usage frequencies as determined for the complete organism of C. glutamicum. Without being wanted to be bound to a theory, it is assumed that the specific codon usage frequency of abundant proteins in an organism such as C. glutamicum reflects certain requirements as to the codon composition of a highly expressed nucleotide sequence.

The specific codon usage distribution of highly expressed genes may e.g. reflect preferences for codons that tire recognised by tRNAs that are also frequently and abundantly available in the host organisms' cells. Similarly such codons may reflect transcript RNA structures that for their spatial arrangement can be more efficiently translated.

Codons that are frequently used in abundant proteins may have been selected for their ability to drive expression. Similarly, codons which are only rarely used in abundant proteins may be prime targets for replacement by other more frequently used codons.

Identifying codon usage frequencies not on the basis of the whole organism, but for abundant proteins thus opens the intriguing possibility of defining specific optimized codon usage information that may be used for overexpression of foreign genes, endogenous genes or mutated versions thereof in a host organism.

It seems reasonable to assume that the finding that highly expressed proteins in a host cell have a different codon usage compared to the situation where, codon usage for all genes of an organism is determined will not be limited to C. glutamicum but also be observed for other organisms such as E. coli, yeast cells, plant cells, insect cells or mammalian cell culture cells.

In view of these surprising findings, the present invention relates to a method of increasing the amount of at least one polypeptide in a host cell comprising the step of expressing a nucleotide sequence for which the codon usage has been adjusted to the codon usage of abundant proteins of the host organism that is used for expression.

In the context of the present invention, the term “increasing the amount of at least one polypeptide in a host cell” refers to the situation that upon expressing the modified nucleotide sequences in the host cell, a higher amount of this polypeptide is produced in a host cell compared to the situation where a non-modified starting nucleotide sequence encoding for a polypeptide of substantially the same amino acid sequence and/or function is expressed in the same type of host cells under similar conditions such as e.g. comparable transfection procedures, comparable expression vectors etc.

The term “host cell” or “organism” for the purposes of the present invention refers to any organism that is commonly used for expression of nucleotide sequences for production of e.g. polypeptides. In particular the term “host cell” or “organism” relates to prokaryotes, lower eukaryotes, plants, insect cells or mammalian cell culture systems.

The organisms of the present invention thus comprise yeasts such as S. pombe or S. cerevisiae and Pichia pastoris.

Plants are also considered by the present invention for overexpressing polypeptides. Such plants may be monocots or dicots such as monocotyledonous or dicotyledonous crop plants, food plants or forage plants. Examples for monocotyledonous plants are plants belonging to the genera of avena (oats), triticum (wheat), secale (rye), hordeum (barley), oryza (rice), panicum, pennisetum, setaria, sorghum (millet), zea (maize) and the like.

Dicotyledonous crop plants comprise inter alias cotton, leguminoses like pulse and in particular alfalfa, soybean, rapeseed, tomato, sugar beet, potato, ornamental plants as well as trees. Further crop plants can comprise fruits (in particular apples, pears, cherries, grapes, citrus, pineapple and bananas), oil palms, tea bushes, cacao trees and coffee trees, tobacco, sisal as well as, concerning medicinal plants, rauwolfia and digitalis. Particularly preferred are the grains wheat, rye, oats, barley, rice, maize and millet, sugar beet, rapeseed, soy, tomato, potato and tobacco. Further crop plants can be taken from U.S. Pat. No. 6,137,030.

Mammalian cell culture systems may be selected from the group comprising e.g. NIH T3 cells, CHO cells, COS cells, 293 cells, Jurkat cells and HeLa cells.

Preferred are microorganisms being selected from the genus of Corynebacterium with a particular focus on Corynebacterium glutamicum, the genus of Escherichia with a particular focus on Escherichia coli, the genus of Bacillus, particularly Bacillus subtilis, and the genus of Streptomyces.

As set out above, a preferred embodiment of the invention relates to the use of host cells which are selected from coryneform bacteria such as bacteria of the genus Corynebacterium. Particularly preferred are the species Corynebacterium glutamicum, Corynebacterium acetoglutamicum, Corynebacterium acetoacidophilum, Corynebacterium callunae, Corynebacterium ammoniagenes, Corynebacterium thermoaminogenes, Corynebacterium melassecola and Corynebacterium effiziens. Other preferred embodiments of the invention relate to the use of Brevibacteria and particularly the species Brevibacterium flavum, Brevibacterium laciofermentum and Brevibacterium divarecatum.

In other preferred embodiments of the invention the host cells may be selected from the group comprising Corynebacterium glutamicum ATCC13032, C. acetoglutamicum ATCC15806, C. acetoacidophilum ATCC13870, Corynebacterium thermoaminogenes FERMBP-1539, Corynebacterium melassecola ATCC17965, Corynebacterium effiziens DSM 44547, Corynebacterium effiziens DSM 44549, Brevibacterium flavum ATCC14067, Brevibacterium lactoformentum ATCC13869, Brevibacterium divarecatum ATCC14020, Corynebacterium glutamicum KFCC10065 and Corynebacterium glutamicum ATCC21608 as well as strains that are derived thereof by e.g. classical mutagenesis and selection or by directed mutagenesis.

Other particularly preferred strains of C glutamicum may be selected from the group comprising ATCC13058, ATCC13059, ATCC13060, ATCC21492, ATCC21513, ATCC21526, ATCC21543, ATCC13287, ATCC21851, ATCC21253, ATCC21514, ATCC21516, ATCC21299, ATCC21300, ATCC39684, ATCC21488, ATCC21649, ATCC21650, ATCC19223, ATCC13869, ATCC21157, ATCC21158, ATCC21159, ATCC21355, ATCC31808, ATCC21674, ATCC21562, ATCC21563, ATCC21564, ATCC21565, ATCC21566, ATCC21567, ATCC21568, ATCC21569, ATCC21570, ATCC21571, ATCC21572, ATCC21573, ATCC21579, ATCC19049, ATCC19050, ATCC19051, ATCC19052, ATCC19053, ATCC19054, ATCC19055, ATCC19056, ATCC19057, ATCC19058, ATCC19059, ATCC19060, ATCC19185, ATCC13286, ATCC21515, ATCC21527, ATCC21544, ATCC21492, NRRL B8183, NRRL W8182, B12NRRLB12416, NRRLB12411, NRRLB12418 and NRRLB11476.

The abbreviation KFCC stands for Korean Federation of Culture Collection, ATCC stands for American-Type Strain Culture Collection and the abbreviation DSM stands for Deutsche Sammlung von Mikroorganismen. The abbreviation NRRL stands for ARS cultures collection Northern Regional Research Laboratory, Peorea, Ill., USA.

Particularly preferred are microorganisms of Corynebacterium glutamicum that are already capable of producing fine chemicals such as L-lysine, L-methionine and/or L-threonine. Therefore the strain Corynebacterium glutamicum ATCC13032 and derivatives of this strain are particularly preferred.

The term “nucleotide sequence” for the purposes of the present invention relates to any nucleic acid molecule that encodes for polypeptides such as peptides, proteins etc. These nucleic acid molecules may be made of DNA, RNA or analogues thereof. However, nucleic acid molecules being made of DNA are preferred.

The terms “non-modified nucleotide sequence” or “starting nucleotide sequence” for the purposes of the present invention, relates to a nucleotide sequence which is intended to be used for (over) expression in a host cell and which has not been amended with respect to its codon usage in the expression host. In case that a foreign polypeptide is to be expressed in the host cell, i.e. a polypeptide with a sequence that is not naturally found within that host cell, the term “non-modified/starting nucleotide sequence” will thus describe e.g. the actual wild-type sequence of that protein. The term “non-modified/starting nucleotide sequence” for the purposes of the present invention may, however, also relate to nucleotide sequences which encode for mutated versions of this protein as long as the nucleotide sequence has not been optimised with respect to the codon usage of abundant proteins in the host cell.

In the embodiment of the present invention wherein an endogenous polypeptide, i.e. a polypeptide that is naturally found within the host cell is to be expressed, the term “non-modified/starting nucleotide sequence” relates to a nucleotide sequence encoding for an endogenous protein or mutated versions thereof which has not been adjusted to the codon usage of abundant proteins of the host cell.

The term “modified nucleotide sequence” for the purposes of the present invention relates to a sequence that has been modified for expression in a host cell by adjusting the sequence of the originally different non-modified/starting nucleotide sequence to the codon usage as used by abundant proteins of the host cell as or by the organism as a whole depending on the context in which this term is used.

The person skilled in the art is clearly aware that modification of the starting nucleotide sequence describes the process of optimization with respect to codon usage.

If, for example, the coding sequence of a foreign wild type enzyme is adjusted to the codon usage of abundant proteins in C. glutamicum, the changes introduced can be easily identified by comparing the modified sequence and the starting sequence which in such a case is the wild type sequence. Moreover, both sequences will encode for the same amino acid sequence.

If, however, the coding sequence of e.g. a foreign or endogenous wild type enzyme is adjusted to the codon usage of abundant proteins in C. glutamicum and if the resulting sequence is simultaneously or subsequently further amended by e.g. deleting amino acids, inserting additional amino acids or introducing point mutations in order to convey e.g. new properties to the enzyme (such as reduced feed back inhibition), the resulting modified nucleotide sequence and modified nucleotide sequence may not encode for identical amino acid sequences. In such a situation, no starting sequence in the sense that the starting sequence and the modified sequence encode for the same amino acid sequence may be present simply because the mutation which has been introduced had not been described before. Nevertheless will a skilled person realize that the inventive method has been used because the starting sequence without the introduced mutation will be known in the form of wild type sequence and the differences of the modified and the starting sequence for those codons which do not code for the introduced mutation will clearly indicate that codon usage optimisation as described above has been carried out. The same applies if mutants in the form of e.g. N-terminal or C-terminal extensions are introduced which have no influence on the function of the protein. Thus, codon usage optimisation will be clear from a comparison of the starting and the modified sequence for those codons which code for the same amino acids at the same or equivalent positions.

This is meant when it is stated in the context of the present invention that the modified and starting nucleotide sequences encode for proteins of substantially identical amino acid sequence. The modified and starting nucleotide sequence will typically be at least 60%, 65%, preferably at least 70%, 75%, 80%, 85% and more preferably at least 90%, 95 or at least 98% identical as regards the amino acid sequence.

The term “abundant proteins” for the purposes of the present invention relates to the group of highly expressed genes within a host cell or organism.

The person skilled in the art is familiar with identifying the group of abundant proteins in a host cell or organism. This may be achieved e.g. by 2D gel electrophoresis. In 2D gel electrophoresis, a protein mixture such as a crude cellular extract is separated on protein gels by e.g. size and isoelectric point. Subsequently these gels are stained and the intensity of the various spot is an indication of the overall amount of protein present in the cell.

Using standard software packages one will select a group of proteins whose signal intensities are above a certain threshold background level and will define this group of protein as abundant proteins. Typical software packages used for this purpose include e.g. Melanie3 (Geneva Bioinformatics SA).

The person skilled in the art is well aware that different host cells such as microorganisms, plant cells, insect cells etc. will differ with respect to the number and kind of abundant proteins in a cell. Even within the same organism, different strains may show a somewhat heterogeneous expression profile on the protein level. One will therefore typically analyse different strains and consider such proteins that are found for all strains to be abundant.

A good selection parameter for defining a group of abundant proteins for the purposes of the present invention is to consider only the 10 to 200 and preferably 10 to 30 most abundant proteins as detected in the above described 2D gel electrophoresis procedure. Preferably one will only consider cytosolic proteins for the group of abundant proteins, only.

Thus, in a preferred embodiment the term “abundant proteins” refers to the group of the approximately 13, 14 or 15 abundant proteins in whole cell cytosolic extracts of host organisms as identified by 2D gel electrophoresis.

Once one has identified the abundant proteins, one may use software tools such as the “Cusp” function of the EMBOSS toolbox version 2.2.0 that can be downloaded at HTTP://EMBOSS.sorceforge.net/download/. Other software packages that may be used are available at www.entelechon.com (e.g. Leto 1.0).

In a preferred embodiment of the present invention, optimising a nucleotide sequence for (over) expression in a host cell by codon usage optimisation may be achieved by modifying the above described starting nucleotide sequence encoding for a polypeptide such that the modified nucleotide sequence uses for each amino acid a more frequently used codon and preferably the most frequently used codon as determined for the group of abundant proteins of a host cell. The modified and the starting nucleotide sequence will encode for substantially the same amino acid sequence and/or function. Both sequences encode for identical amino acid sequences at least at those positions which have been optimized for codon usage. This does not preclude that additional mutations as described may be introduced into the modified sequences.

In another preferred embodiment “rare”, “very rare”, “extremely rare” and preferably the least frequently used codons of the non-modified sequence will be replaced by “frequent” codons in the modified sequence with codon frequency being determined for the group of abundant proteins of the respective host cell.

Unless otherwise indicated the terms “rare” and “frequent” codons refer to the relative frequency by which a certain codon of all possible codons encoding a specific amino acid is used by the group of abundant proteins.

A codon will be considered to be “rare” if it is used less than 20% for the specific amino acid. A “very rare” codon will be used at a frequency of less than 10% and an “extremely rare” codon will be used at a frequency of less than 5%. The frequency is determined on the basis of the codon usage of the abundant proteins of the host organism.

As the amino acids methionine and tryptophane are encoded by one codon only, the respective codon frequency is always 100%. However the amino acid alanine is encoded by four codons, namely GCU, GCC, GCA and GCG. For the whole organism of C. glutamicum these codons are used at a relative frequency of 23.7%, 25.4%, 29.3% and 21.6% (see Table 1, experiment 1). However, in the group of abundant proteins, these codons are used at relative frequencies of 46.8%, 9.9%, 35.9% and 7.4% (see Table 2, experiment 1).

In view of the above explanations the codons GCC and GCG are thus considered to be rare and more precisely to be very rare codons. The replacement of “rare”, “very rare” and “extremely rare” codons can prove beneficial because “brakes” of translational efficiency are removed.

Similarly a codon will be considered to be “frequent” if it used at a relative frequency of more than 40%. It is “very frequent” if is used at relative frequency of more than 60% and a relative frequency of more than 80% is indicative of an “extremely frequent” codon. Again the relative frequencies are based, on the codon usage of the abundant proteins of the host cell unless otherwise indicated.

The terms “express”, “expressing”, “expressed” and “expression” refer to expression of a gene product (e.g., a biosynthetic enzyme of a gene of a pathway) in a host organism. The expression can be done by genetic alteration of the microorganism that is used as a starting organism. In some embodiments, a microorganism can be genetically altered (e.g., genetically engineered) to express a gene product, at an increased level relative to that produced by the starting microorganism or in a comparable microorganism which has not been altered. Genetic alteration includes, but is not limited to, altering or modifying regulatory sequences or sites associated with expression of a particular gene (e.g. by adding strong promoters. Inducible promoters or multiple promoters or by removing regulatory sequences such that expression is constitutive), modifying the chromosomal location of a particular gene, altering nucleic acid sequences adjacent to a particular gene such as a ribosome binding site or transcription terminator, increasing the copy number of a particular gene, modifying proteins (e.g., regulatory proteins, suppressors, enhancers, transcriptional activators and the like) involved in transcription of a particular gene and/or translation of a particular gene product, or any other conventional means of deregulating expression of a particular gene using routine in the art (including but not limited to use of antisense nucleic acid molecules, for example, to block expression of repressor proteins).

Overexpression for the purposes of the present, invention means that the amount of the polypeptide that is to be overexpressed is increased by at least 5%, 10%, 20%, 30%, 40%, 50%, 60%, 70%, 80%, 90% or 100% and preferably by a factor of at least 3, 4, 5, 6, 7, 8, 9 or 10 and more preferably by a factor of at least 20, 50, 100, 500 or 1000 if expression of the modified nucleotide sequence is compared to expression of the starling nucleotide sequence in the same type of host organism under a comparable situation (comparable chromosomal position of the respective sequences, comparable vectors, comparable promoters etc.).

The method of the present invention may be used to (over)express polypeptides from an organism different than the host cell, i.e. foreign polypeptides as mentioned above. Foreign polypeptides will be encoded by nucleotide sequences that are naturally not found in the host cell. Thus, expression of foreign polypeptides relates to the situation where e.g. an enzyme is expressed with the enzymatic activity thereof not at all being present in the host organism or it may refer to a situation where a homolog of a host-specific factor is expressed. One may for example express a homolog of a certain enzyme derived from E. coli in C. glutamicum.

Another embodiment of the present invention uses the inventive method for (over) expressing endogenous polypeptides of the host cell, i.e. polypeptides being encoded by sequences that are naturally found within the host cell. For example, a host cell-specific low-abundance protein may be overexpressed by modifying the different starting nucleotide sequence encoding for the low-abundance protein such that the codon usage of the modified nucleotide sequence which in tins case may encode for a polypeptide of identical amino acid is adjusted to the codon usage of abundant proteins of the host cell as defined above.

In some embodiments it may be sufficient and preferred to replace rare, very rare and extremely rare codons with more frequently used codons as determined for the group of abundant proteins. In a preferred embodiment the modified nucleotide sequence may use for each of the least frequently used codon the most frequently used codon of the abundant proteins of the host cell.

The above methods may be used for production of fine chemicals as defined below. To this end, the modified nucleotide sequences may be selected from genes which are known to participate in the biosynthesis of such fine chemicals. Particularly preferred are genes for which overexpression is known to stimulate fine chemical production.

A preferred method in accordance with the present invention relates to a method of increasing the amount of at least one polypeptide in Corynebacteria and preferably in C. glutamicum comprising the step of expressing a modified nucleotide sequence coding for at least one polypeptide on Corynebacteria and preferably in C. glutamicum wherein said modified nucleotide sequence is derived from a different starting sequence such that the codon usage of the modified nucleotide sequence is adjusted to the codon usage of abundant proteins of Corynebacterium in general and preferably of C. glutamicum. The modified and starting nucleotide sequence will encode for substantially the same and in some cases identical amino acid sequences as described above. The modified and starting nucleotide sequences will thus encode substantially the same amino acid sequence and/or function. Both sequences encode for identical amino acid sequences at least at those positions which have been modified in the course of codon usage adaption.

Of course, the definitions as provided above for the meaning of the terms “modified nucleotide sequences”, “starting (non-modified) nucleotide sequences”, “rare codons”, “frequent codons” etc. apply equally for these preferred embodiments of the invention.

The abundant proteins of e.g. C. glutamicum can be determined as described above by 2D protein gel electrophoresis. To this purpose, C, glutamicum strains may be cultivated under standard conditions. Then, cell extracts may be prepared using common lysis protocols. After lysis, the cell extracts are centrifuged and approximately 25-50 μg are analyzed by standard 2D-PAGE. An example of the approach can be found below in example 1 as well as in the material and methods part of Hansmeier et al. (Proteomics 2006, 6, 233-250)

Following this approach abundant proteins in C. glutamicum can be identified by either selecting the most abundant 10 to 300 cytosolic proteins or by identifying 10 to 30 cytosolic proteins that are observed to be present in elevated amounts in various strains. These results are assumed to be representative also for the group of abundant proteins in other Corynebacterium species.

For the purposes of the present invention, the term “abundant proteins of C. glutamicum” can relate to the group comprising the following protein factors (accession number of nucleotide sequence shown in brackets):
Elongation factor Tu (Genbank accession no: X77034)
Glycerin-aldehyde-3-phosphate-dehydrogonase (Genbank accession no; BX927152, ±, nt. 289401-288397)
Fructose bisphosphate aldolase (Genbank accession no: BX927156, ±, nt. 134992-133958)
Elongation Factor Ts (Genbank accession no: BX927154, ±, nt. 14902-14075)
Hypothetical protein (Genbank accession no: BX927155, ±, nt. 213489-214325)
Enolase (Genbank accession no: BX927150, nt. 338561-339838)
Peptidyl-prolyl-Cis-trans isomerase (Genbank accession no: BX927148, nt. 34330-34902)
Superoxide dismutase (Genbank accession no: AB055218)
Phosphoglycerate dehydrogenase (Genbank accession no: BX92715L nt. 306039-307631)
SSU Rib protein SIP (Genbank accession no: BX927152, ±, nt. 26874-28334)
Triose phosphate-isomerase (Genbank accession no: BX927152, ±, nt. 286884-286105)
Isopropylmalat-synthase (Genbank accession no; X70959)
Butane-2,3-dioldehydrogenase (Genbank accession no: BX927156, nt. 20798-21574)
Fumarate-hydratase (Genbank accession no: BX927151, ±, nt. 18803-17394)

On the basis of these aforementioned fourteen proteins, a codon usage table can be created using the aforementioned “CUSP” function of the EMBOSS toolbox.

The above-described group of fourteen proteins may particularly be used for determining or for defining the group of abundant proteins in C. glutamicum if the C, glutamicum strain ATCC 13032 and/or derivatives (obtained e.g. by classical mutagenesis and selection or genetic engineering) are used in the 2D-gel electrophoresis analysis.

Using the CUSP function of the EMBOSS toolbox version one can thus create a Codon Usage Table that reflects codon usage of abundant proteins of Corynebacterium in general and preferably of C. glutamicum.

Surprisingly the codon usage of these abundant proteins differs significantly from the codon usage as determined for the whole genome of C. glutamicum as becomes clear from a comparison of Tables 1 and 2 (see Experiment 1 below). Codon usage of the whole genome of C. glutamicum can e.g. be determined from strains that are completely sequenced such as strain ATCC13032 and Codon Usage Tables may e.g. generated by the CUSP function of the aforementioned EMBOSS toolbox or are available at e.g. HTTP://www.kazusa.or.jp. Highly-comparable results are obtained if one uses the most abundant cytosolic proteins as mentioned in Table 4 of Hansmeier et al. (vide supra).

Thus, a preferred embodiment of the invention relates to a method of increasing the amount of polypeptides in Corynebacteria and particularly in C. glutamicum by expressing a modified nucleotide sequence which is derived from a different starting nucleotide sequence with the modified nucleotide sequence being adjusted to the codon usage of Table 2.

In yet another embodiment which is also preferred, the codons of the modified nucleotide sequence are selected for at least one and preferably for each amino acid from one of the two most frequently used codons as set forth in Table 2. If there are less than three codons encoding an amino acid, only the most frequently used codon of Table 2 should be used.

Similarly a preferred embodiment of the invention relates to the use of modified nucleotide sequences for increasing the amount of a polypeptide in Corynebacteria and particularly in C. glutamicum wherein the codons GUU for valine, GCU for alanine, GAC for aspartic acid, GAG for glutamic acid and/or ATG for methionine (if it is the start codon) are used.

In a particularly preferred embodiment the method of increasing the amount of a polypeptide in Corynebacteria and particularly in C. glutamicum comprises the step of expressing a modified nucleotide sequence having been derived from a starting nucleotide sequence wherein the codons of the modified nucleotide sequence are selected for at least one, some and preferably for each amino acid from the codon usage of Table 3.

In all of the aforementioned methods for increasing expression of a polypeptide in Corynebacterium and preferably in C. glutamicum, rare, very rare codons and extremely rare codons are preferably exchanged against more frequently used and preferably the most frequently used codons as they can be taken from Table 2. While exchange of one rare, very rare or extremely rare codon against a more frequently used codon of e.g. Table 2 may already lead to an increased expression, it may be preferred to exchange more than one rare, very rare or extremely codons up to all rare, very rare or extremely codons.

Of course, the methods that are used to increase the expression of a polypeptide in Corynebacteria and particularly in C. glutamicum may be used to express foreign polypeptides or endogenous polypeptides of Corynebacteria and particularly of C. glutamicum. The methods in accordance with the invention may also comprise to overexpress modified sequences which haven been further amended by inserting or deleting amino acids or in which point mutations have been introduced.

In a preferred embodiment the above described methods in which the modified nucleotide sequence is e.g. adapted to the codon usage of Table 2 or in which the modified nucleotide sequences uses the two most frequently used codons of Table 2, the codons of Table 3 or the aforementioned codons for valine, alanine, aspartic acid, glutamic acid and/or the start methionine, the host organism may be selected from the group comprising Corynebacterium glutamicum, Corynebacterium acetoglutamicum, Corynebacterium acetoacidophilum, Corynebacterium thermoaminogenes, Corynebacterium melassecola and Corynebacterium effiziens.

Also preferred are the above-mentioned C. glutamicum strain and particularly preferred is the strain Corynebacterium glutamicum ATCC13032 and all its derivatives. The strains ATCC 13286, ATCC 13287, ATCC 21086, ATCC 21127, ATCC 21128, ATCC 21129, ATCC 21253, ATCC 21299, ATCC 21300, ATCC 21474, ATCC 21475, ATCC 21488, ATCC 21492, ATCC 21513, ATCC 21514, ATCC 21515, ATCC 21516, ATCC 21517, ATCC 21518, ATCC 21528, ATCC 21543, ATCC 21544, ATCC 21649, ATCC 21650, ATCC 21792, ATCC 21793, ATCC 21798, ATCC 21799, ATCC 21800, ATCC 21801, ATCC 700239, ATCC 21529, ATCC 21527, ATCC 31269 and ATCC 21526 which are known to produce lysine can also preferably be used. The other aforementioned strains can also be used.

In another embodiment of the invention, a vector that comprises the aforementioned nucleotide sequences is used to drive expression of a modified nucleotide sequence in the host cell, preferably in Corynebacterium and particularly preferably in C. glutamicum for increasing the amount of a polypeptide in these host cells. Such vectors may e.g. be plasmid vectors which are autonomously replicable in coryneform bacteria. Examples are pZ1 (Menkel et al. (1989), Applied and Environmental Microbiology 64: 549-554), pEKEx1 (Eikmanns et al. (1991), Gene 102: 93-98), pHS2-1 (Sonnen et al. (1991), Gene 107: 69-74) These vectors are based on the cryptic plasmids pHM1519, pBL1 or pGA1. Other vectors are pCLiK5MCS (WO2005059093), or vectors based on pCG4 (U.S. Pat. No. 4,489,160) or pNG2 (Serwold-Davis et al. (1990), FEMS Microbiology Letters 66, 119-124) or pAG1 (U.S. Pat. No. 5,158,891).

When optimizing the codon usage, other influencing factors, like the resulting mRNA structure, should also be considered. One should e.g. possibly avoid to generate a mRNA secondary structure which is unstable. Furthermore on should possibly avoid using a codon recognized by the same tRNA in direct physical proximity on the mRNA.

Of course, the above described preferred embodiments of the invention which relate to methods of increasing the amount of a polypeptide in a host cell by using codon usage optimised nucleotide sequences which have been obtained with respect to the codon usage of the above defined group of the approximately fourteen most abundant proteins in C. glutamicum can be used to overexpress polypeptides being encoded by the modified nucleotide sequence in C. glutamicum.

With overexpression it is meant that the amount of the at least polypeptide is increased by at least 5%, 10%, 20%, 30%, 40%, 50%, 60%, 70%, 80%, 90% or 100% and preferably by a factor of at least 3, 4, 5, 6, 7, 8, 9 or 10 and more preferably by a factor of at least 20, 50, 100, 500 or 1000 if expression of the modified nucleotide sequence is compared to expression of the starting nucleotide sequence under comparable conditions.

It is understood that it is not always desirable to increase expression as much as possible. In certain cases an increase of factor 3 may be sufficient and desirable. The present invention offers the possibility to fine tune repression by e.g. not replacing all codons by the most frequently used codons, but by e.g. exchanging only two or three (rare) codons at selected positions.

A preferred embodiment of the present invention relates to methods of increasing the amount of a polypeptide in a host cell, preferably in Corynebacterium and more preferably in C. glutamicum wherein the above described modified nucleotide sequences are selected from the group comprising nucleotide sequences encoding genes of biosynthetic pathways of fine chemicals for which overexpression is known to enhance production of the fine chemicals.

The term “fine chemical” is well known to the person skilled in the art and comprises compounds which can be used in different parts of the pharmaceutical industry, agricultural industry as well as in the cosmetics, food and feed industry. Fine chemicals can be the final products or intermediates which are needed for further synthesis steps. Fine chemicals also include monomers for polymer synthesis.

Fine chemicals are defined as all molecules which contain at least two carbon atoms and additionally at least one heteroatom which is not a carbon or hydrogen atom. Preferably fine chemicals relate to molecules that comprise at least two carbon atoms and additionally at least one functional group, such as hydroxy-, amino-, thiol-, carbonyl-, carboxy-, methoxy-, ether-, ester-, amido-, phosphoester-, thioether- or thioester-group.

Fine chemicals thus preferably comprise organic acids such as lactic acid, succinic acid, tartaric acid, itaconic acid etc. Fine chemicals further comprise amino acids, purine and pyrimidine bases, nucleotides, lipids, saturated and unsaturated fatty acids such as arachidonic acid, alcohols, e.g. diols such as propandiol and butandiol, carbohydrates such as hyaluronic acid and trehalose, aromatic compounds such as vanillin, vitamins and cofactors etc.

A particularly preferred group of fine chemicals for the purposes of the present invention are biosynthetic products being selected from the group comprising organic acids, proteins, amino acids, lipids etc. Other particularly preferred line chemicals are selected from the group of sulphur containing compounds such as thionine, cysteine, homocysteine, cystathionine, glutathione, biotine, thiamine and/or lipoic acid.

The group of most preferred line chemical products include amino acids among which glycine, lysine, methionine, cysteine and threonine are particularly preferred.

In a preferred embodiment of the present invention the method for increasing the amount of a polypeptide in a Corynebacterium such as C. glutamicum uses modified nucleotide sequences for which codon usage has been optimized as described above and which, have been derived from starting nucleotide sequences selected from the group comprising sequences encoding aspartate kinase, aspartate-semialdehyde-dehydrogenase, diaminopimelate-dehydrogenase, diaminopimelate-decarboxylase, dihydrodipicolinate-synthetase, dihydrodipicolinate-reductase, pyruvate carboxylase, transcriptional regulators LuxR, transcriptional regulators LysR1, transcriptional regulators LysR2, malate-quinone-oxidoreductase, glucose-6-phosphate-dehydrogenase, 6-phosphogluconate-dehydrogenase, transketolase, transaldolase, lysine-exporter, arginyl-t-RNA-synthetase, phosphoenolpyruvate-carboxylase, fructose-1,6-bisphosphatase, protein OpcA, 1-phosphofructokinase, 6-phosphofructokinase, biotin-ligase, tetrahydropicolinat-succinylase, succinyl-aminoketopimelate-aminotransferase, succinyl-diaminopimelate-desuccinylase, diaminopimelate-epimerase, 6-phosphogluconate-dehydrogenase, gucosephosphate-isomerase, phosphoglycerate-mutase, pyruvate-kinase, aspartate-transaminase and malate-enzyme.

In a particularly preferred embodiment of the present invention the method for increasing the amount of a polypeptide in a Corynebacterium such as C. glutamicum uses modified nucleotide sequences for which codon usage has been optimized as described above and which have been derived from starting nucleotide sequences selected from the group comprising sequences encoding aspartate-kinase, aspartate-semialdehyde-dehydrogenase, homoserine-dehydrogenase, glycerinaldehyde-3-phosphate-dehydrogenase, 3-phosphoglycerate-kinase, pyruvate-carboxylase, homoserine-O-ccetyltransferase, cystahionine-gamma-synthase, cystahionine-beta-lyase, serine-hydroxymethyltransferase, O-acetylhomoserine-sulfhydrylase, methylene-tetrahydrofolate-reductase, phosphoserine-aminotransferase, phosphoserine-phosphatase, serine-acetyl-transferase, cysteine-synthase, cysteine-synthase II, coenzyme B12-dependent methionine-synthase (metH), coenzym B12-independent methionine-synthase, sulfate-adenylyltransferase, phosphoadenosins-phosphosultate-reduetase, ferredoxine-suifite-reductase, ferredoxine-NADPH-reductase, ferredoxine-protein activity of sulfate-reduction RXA077, protein activity of sulfate-reduction RXA248, protein activity of sulfate-reduction RXA247, protein activity of RXA655-regulator and protein activity of RXN2910-regulator, 6-phosphogluconate-dehydrogenase, glucosephosphate-isomerase, phosphoglycerate-mutase, pyruvate-kinase, aspartate-transaminase, malate-enzyme, dihydrodipicolinate-synthetase, dihydridipicolinate-reductase, diaminopimelate-dehydrogenase, diaminopimelate-decarboxylase, lysine-exporter, pyruvate carboxylase, phosphoenolpyruvate (PEP) carboxylase, glucose-6-phosphate-dedyrogenase, 6-phospho-gluconolactonase, ribose-5-phosphate-isomerase, ribose-phosphate epimerase, transketolase, transaldolase, glucosephosphate-isomerase, transcriptional regulators LuxR, transcriptional regulators LysR1, transcriptional regulators LysR2, malate-quinone-oxidoreductase, malate dehydrogenase, fructose-1,6-bisphosphatase triosephosphate isomerase, glyceraldehyde-phosphate.dehydrogenase, phosphoglycerate kinase, phosphoglycerate mutase, enolase, pyruvate kinase, arginyl-t-RNA-synthetase, protein OpcA, 1-phosphofructokinase, 6-phosphofructokinase, biotin-ligase, isocitrate lyase, malate synthase, tetrahydropicolinat-succinylase, succinyl-aminoketopimelate-aminotransferase, succinyl-diaminopimelate-desuccinylase, diaminopimelate-epimerase, aspartate-transaminase, components of the PTS sugar uptake system, accBC (acetyl CoA carboxylase), accDA (acetyl CoA carboxylase), aeeA (isocitrat-lyase), acp (acyl carrier protein), asp (aspartase), atr61 (ABC transporter), cesB (cytochrome synthesis protein), edsA (phosphatidat-cytidyltransferase), citA (sensor kinase of a 2-component system), els (cardiolipin synthase), cma (cyclopropane-myolic acid synthase), cobW (cobalamin synthesis-related protein), cstA (carbon starvation protein A), ctaD (Cytocrom aa3 Oxidase UE1), ctaE (cytocrom aa3 oxidase UE3), ctaF, 4 (subunit of cytochrome aa3 oxidase), cysD (sulfate-adenosyltransferase), cysE (serine-acetyltransferase, cysH, cysK (cysteine synthase), cysN (sulfat-adenosyltransferase), cysQ transport protein), dctA (C4 dicarboxylate transport protein), dep67 (cobalamin synthesis-related protein), dps (DNA protection protein), dtsR (propionyl-CoA carboxylase), fad15 (acyl-CoA-synthase), ftsX (cell division protein), glbO (HB-like protein), glk (glukokinase), gpmB (phosphoglycerate kinase II), hemD hemB (uroporphyrinogen-II-synthase, delta-aminolevulinic acid dehydratase), lldd2 (lactate dehydrogenase), metY (O-acetylhomoserine-sulfhydrylase), msiK (sugar Import protein), ndkA (nucleoside diphosphate kinase), nuoU (NADH-dehydrogenase subunit V), nuoV (NADH-dehydrogenase subunit V), nuoW (NADH-dehydrogenase subunit W), oxyR (transcriptional regulator), pgsA2 (CDP-diacylglycerol-3-P-3-phosphatidyltransferase), pknB (protein kinase B), pknD (protein kinase D), plsC (1-Acyl-SN-glycerol-3-P-acyltransferase), poxB and (pyruvat oxidase, 6-phosphoglucnonate dehydrogenase), ppgK (polyphosphate glucokinase) ppsA (PEP synthase), qcrA (Rieske Fe-S-protein), qcrA (Rieske Fe-S-protein), qcrB (cytochrom B), qcrB (cytochrom B), qcrC (cytochrom C), rodA (cell division protein), rpe (ribulose phosphate isomerase), rpi (phosphopentose isomerase), sahH (adenosyl homocysteinase), sigC (sigma factor C), sigD (activator of transcription factor sigma D), sigE (sigma factor E), sigh (sigma factor H), sigM (sigma factor M), sod (superoxiddismutase), thyA (thymidylate synthase), truB (tRNA pseudouridine 55 synthase) and zwa1 (PS1-protein), These sequences and methods may be used to particularly obtain lysine.

In a further particularly preferred embodiment of the present invention the method for increasing the amount of a polypeptide in a Corynebacterium such as C. glutamicum uses modified nucleotide sequences for which codon usage has been optimized as described above and which have been derived from starting nucleotide sequences selected from the group comprising sequences encoding aspartate-kinase, aspartate-semialdehyde-dehydrogenase, glycerinaldehyde-3-phosphate-dehydrogenase, 3-phosphoglycerate-kinase, pyruvate-carboxylase, triosephosphate-isomerase, threonine-synthase, threonin-export-carrier, transaldolase, transketolase, glucose-6-phosphate-dehydrogenase, malate-quinone-oxidoreductase, homoserine-kinase, biotine-ligase, phosphoenolpyruvate-carboxylase, threonine-efflux activity, protein OpcA, 1-phosphofructo-kinase, 6-phosphofructo-kinase, fructose-1,6bisphosphatase, 6-phosphogluconate-dehydrogenase, homoserine-dehydrogenase 6-phosphogluconate-dehydrogenase, phosphoglycerate-mutase, pyruvat-kinase, aspartate-transaminase and malate-enzyme. These sequences and methods may be particularly be suited to obtain methionine.

In a further particularly preferred embodiment of the present invention the method for increasing the amount of a polypeptide in a Corynebacterium such as C. glutamicum uses modified nucleotide sequences for which codon usage has been optimized as described above and which have been derived from stalling nucleotide sequences selected from the group comprising sequences encoding dehydratase, homoserin O-ccetyltransferase, serine-hydroxymethyltransferase, O-acetylhomoserine-sulfhydrylase, meso-siaminopimelate-D-dehydrogenase, phosphoenoipyruvate-carboxykinase, pyruvat-oxidase, dihydrodipicolinate-synthetase, dihydrodipicolinate-reductase, asparaginase, aspartate-decarboxylase, lysine-exporter, acetolactate-synthase, ketol-acid-reductoisomerase, branched chain aminotransferase, coenzyme B12-dependent methionine-synthase (metH), coenzym B12-independent methionine-synthase, di hydroxy acid-dehydratase and diaminopicolinate-decarboxylase. These sequences and methods may be particularly be suited to obtain threonine.

Another aspect of the present invention relates to the modified nucleotide sequences which encode for a polypeptide allowing for increased expression of the polypeptide in a host cell wherein the modified nucleotide sequence is derived from a different starting nucleotide sequence with the codon usage of the modified nucleotide sequence being adjusted to the codon usage of the abundant proteins of the respective host cells. The modified and starting nucleotide sequence encode for substantially the same amino acid sequence and/or function. Both sequences usually encode for identical amino acid sequences at least at the positions where they have been modified with respect to codon usage.

The definitions given above as to the meaning of host cell, abundant proteins and how to determine them equally apply.

Preferred embodiments also relate to modified nucleotide sequences which are to be used for expression of a polypeptide in the host cell and wherein the modified nucleotide sequences have been derived from a starting sequence by adjusting the codon usage of the modified nucleotide sequence to the codon usage of abundant proteins of the genus Corynebacterium and preferably of C. glutamicum. The modified and starting nucleotide sequence encode for substantially the same amino acid sequence and/or function. Both sequences usually encode for identical amino acid sequences at least at the positions where they have been modified with respect to codon usage.

Again, for the purposes of this aspect of the invention, the definitions given above equally apply so that the term abundant proteins in e.g. C. glutamicum will essentially relate to the same group of fourteen proteins mentioned above.

Yet another preferred embodiment of this invention relates to modified nucleotide sequences for expression in Corynebacterium and preferably in C. glutamicum wherein the codon usage of the modified nucleotide sequence has been adjusted to the codon usage of Table 2. Similarly, other preferred embodiments relate to nucleotide sequence wherein the codons of the modified nucleotide sequence are selected for at least one and preferably for each amino acid from one of the two most frequently used codons of Table 2.

In addition or alternatively, the modified nucleotide sequences for expression of polypeptides in Corynebacterium and preferably in C. glutamicum may use the codons GUU for valine, GCU for alanine, GAC for aspartic acid, GAG for glutamic acid and/or ATG for the start methionine.

A particularly preferred embodiment of the present invention relates to a modified nucleotide sequence that is used to drive expression of a polypeptide in Corynebacterium and preferably in C. glutamicum wherein the codons of the modified nucleotide sequence wherein the codons have been selected for at least one and preferably for each amino acid from the codon usage of Table 3.

In all of the aforementioned modified nucleotide sequences for increasing expression of a polypeptide in Corynebacterium and preferably in C. glutamicum, rare codons, very rare codons and extremely rare codons are preferably exchanged by more frequently used and preferably the most frequently used codons as they can be taken from Table 2. While exchange of one (rare) codon against a more frequently used codon of Table 2 may already lead to an increased expression, it is preferred to exchange more and preferably all rare codons.

These modified nucleotide sequences may again be preferably selected from the group comprising nucleotide sequences encoding genes of biosynthetic pathways of fine chemicals. The definitions and preferences as to the meaning and desirability of fine chemicals given above equally apply.

As a consequence, in the case of producing the fine chemical lysine the nucleotide sequence may be selected from the group comprising the aforementioned sequences. The same applies if the fine chemicals methionine and threonine are to be produced.

Further aspects of the invention relate to vectors that are suitable for expression of a polypeptide in a host cell wherein the vector comprises the aforementioned nucleotide sequences. Of course, a preferred embodiment will relate to vectors that are capable of driving expression of polypeptides in microorganisms such as Corynebacterium and preferably such as C. glutamicum.

Host cells comprising the aforementioned nucleotide sequences or vectors also form part of the invention with host cells derived from Corynebacterium and particularly from C. glutamicum being preferred.

Other aspects of the invention relate to the use of methods as put forward above, to the use of nucleotide sequences as put forward above, to the use of a vector as put forward above and to the use of a host cell as put forward above for producing the aforementioned fine chemicals.

In preferred embodiments of the invention, one will use the codon usage optimised nucleotide sequences that allow to drive expression in a host such as Corynebacterium and preferably C. glutamicum and that have been codon-usage optimised with respect to the abundant proteins of e.g. C. glutamicum.

In general, the person skilled in the art is familiar with designing constructs such as vectors for driving expression of a polypeptide in microorganisms such as E. coli and C. glutamicum. The person skilled in the art is also well acquainted with culture conditions of microorganisms such as C. glutamicum and E. coli as well as with procedures for harvesting and purifying fine chemicals such as amino acids and particularly lysine, methionine and threonine from the aforementioned microorganisms. Some of these aspects will be set out in further detail below.

The person skilled in the art is also well familiar with techniques that allow to change the original starting nucleotide sequence into a modified nucleotide sequence encoding for polypeptides of identical amino acid but with different codon usage. This may e.g. be achieved by polymerase chain reaction based mutagenesis techniques, by commonly known cloning procedures, by chemical synthesis etc. Some of these procedures are set out in the examples.

Another embodiment of the present invention relates to a method of increasing the amount of a polypeptide in a host cell by expressing a modified nucleotide sequence which has been amended with respect to the codon usage of the abundant proteins of the host cell.

However, other than described above where codons have been replaced in the original different non-modified sequence by selecting the more frequently used codons of the group of abundant proteins of the host cell, the modified sequences are not optimised in this way, but rather are obtained by replacing codons in the original different non-modified nucleotide sequences with codons that axe used in the group of abundant proteins at a similar distribution frequency. If for example the original nucleotide sequence uses the codon CUU at a frequency of 10% and the codon CUA at a frequency of 50% and if e.g. in the group of abundant proteins the codon CUC is used at a frequency of 20% and the codon CUG is used at a frequency of 60%, in the modified nucleotide sequence the codon CUU will be replaced by CUC and the codon CUA will be replaced by CUG. Thus, in one embodiment of the present invention methods for increasing the amount of a polypeptide in a host cell does not aim that much at optimising coding usage in terms of overall frequency but instead of harmonising the distribution frequency throughout die coding sequence.

Yet another embodiment of the present invention relates to methods for increasing the amount of polypeptides in Corynebacterium and particularly preferably in C. glutamicum in which a modified nucleotide sequence is expressed wherein the sequence of the modified nucleotide sequence has been adjusted to the codon usage of the complete organism of C. glutamicum as set forth in Table 1. Of course, in methods relating to this aspect of the invention a modified nucleotide sequence may be used, wherein at least one, at least two, at least three, at least four, at least, five, at least six, at least seven, at least eight, at least nine, at least ten, preferably at least 1%, at least 2%, at least 4%, at least 6%, at least 8%, at least 10%, more preferably at least 20%, at least 40%, at least 60%, at least 80%, even more preferably at least 90% or least 95% and most preferably all of the codons of the starting nucleotide sequence are replaced in the resulting modified nucleotide sequence by more frequently and preferably the most frequently used codons for the respective amino acid according to Table 1. In an even more preferred embodiment the afore-mentioned number of codons to be replaced refers to rare, very rare and particularly extremely rare codons.

The above given definitions of “modified nucleotide sequence”, “starting/non-modified nucleotide sequences”, “host cells” etc. as well as the explanations given e.g. for the achievable extent of expression equally apply if modification is based on the codon usage as determined for the whole organism. The definitions of rare, very rare and extremely rare as well as of frequent, very frequent and extremely frequent codons equally apply except that the relative frequency of a codon is not determined on the basis of abundant proteins but on the basis of the codon usage of the organism. Of course, the present invention also relates to modified nucleotide sequences the codon usage of which has been adjusted to the codon usage of the organism of C. glutamicum as put forward in Table 1. Similarly, the present invention relates to expression vectors which can be used to express such nucleotide sequences in C. glutamicum and host cells comprising such sequences and vectors. The host cells can be selected from the C. glutamicum strains as mentioned above. As far as optimisation of codon usage is based on C. glutamicum as an organism, the present invention also relates to the use of such methods, modified nucleotide sequences, vectors and host cells for producing fine chemicals. The production of fine chemicals such a amino acids and particularly lysine, methionine and tryptophane is preferred in this context. For the production of these fine chemicals, the starting nucleotide sequences may be selected from factors which are involved in the biosynthesis of these compounds and particularly from the above mentioned lists.

In the following, it will be described and set out in detail how genetic manipulations in microorganisms such as E. coli and particularly Corynebacterium glutamicum can be performed.

### Vectors and Host Cells

One aspect of the invention pertains to vectors, preferably expression vectors, containing a modified nucleotide sequences as mentioned above. As used herein, the term “vector” refers to a nucleic acid molecule capable of transporting another nucleic acid to which it has been linked.

One type of vector is a “plasmid”, which refers to a circular double stranded DNA loop into which additional DNA segments can be ligated, Another type of vector is a viral vector, wherein additional DNA segments can be ligated into the viral genome.

Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked.

Such vectors are referred to herein as “expression vectors”.

In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent, functions.

The recombinant expression vectors of the invention may comprise a modified nucleic acid as mentioned above in a form suitable for expression of the respective nucleic acid in a host cell, which means that, the recombinant expression vectors include one or more regulatory sequences, selected on the basis of the host cells to be used for expression, which is operatively linked to the nucleic acid sequence to be expressed.

Within a recombinant expression vector, “operably linked” is intended to mean that the nucleotide sequence of interest is linked to the regulatory sequence (s) in a manner which allows for expression of the nucleotide sequence (e.g., in an in vitro transcription/translation system or in a host cell when the vector is introduced into the host cell). The term “regulatory sequence” is intended to include promoters, repressor binding sites, activator binding sites, enhancers and other expression control elements (e.g., terminators, polyadenylation signals, or other elements of mRNA secondary structure). Such regulatory sequences are described, for example, in Goeddel; Gene Expression Technology: Methods in Enzymology 185, Academic Press, San. Diego, Calif. (1990). Regulatory sequences include those which direct constitutive expression of a nucleotide sequence in many types of host cell and those which direct expression of the nucleotide sequence only in certain host cells. Preferred regulatory sequences are, for example, promoters such as cos-, tac-, trp-, tet-, tip-, let-, lpp-, lac-, lpp-lac-, lacIq-, T7-, T5-, T3-, gal-, trc-, ara-, SP6-, arny, SP02, e-Pp-ore PL, SOD, EFTu, EFTs, GroEL, MetZ (all from C. glutamicum), which are used preferably in bacteria. Additional, regulatory sequences are, for example, promoters from yeasts and fungi, such as ADC1, MFa, AC, P-60, CYC1, GAPDH, TEF, rp28, ADH, promoters from plants such as CaMV/35S, SSU, OCS, lib4, usp, STLS1, B33, nos or ubiquitin- or phaseolin-promoters. It is also possible to use artificial promoters. It will be appreciated by one of ordinary skill in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by the above-mentioned modified nucleotide sequences.

The recombinant expression vectors of the invention can be designed for expression of the modified nucleotide sequences as mentioned above in prokaryotic or eukaryotic cells. For example, the modified nucleotide sequences as mentioned above can be expressed in bacterial cells such as C. glutamicum and E. coli, insect cells (using baculovirus expression vectors), yeast and other fungal cells (see Romanos, M. A. et al. (1992), Yeast 8: 423-488; van den Hondel, C. A. M. J. J. et al. (1991) in: More Gene Manipulations in Fungi. J. W. Bennet & L. L, Lasure, eds., p. 396-428: Academic Press: San Diego; and van den Hondel, C. A. M. J. J. & Punt, P. J. (1991) in: Applied Molecular Genetics of Fungi, Peberdy, J. F. et al., eds., p. 1-28, Cambridge University Press: Cambridge), algae and multicellular plant cells (see Schmidt, R. and Willmitzer, L. (1988) Plant Cell Rep.: 583-586). Suitable host cells are discussed further in Goeddel, Gene Expression Technology: Methods in Enzymology 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out with vectors containing constitutive or inducible promoters directing the expression of either fusion or non-fusion proteins.

Fusion vectors add a number of amino acids to a protein encoded therein, usually to the amino terminus of the recombinant protein but also to the C-terminus or fused within suitable regions in the proteins. Such fusion vectors typically serve four purposes; 1) to increase expression of recombinant protein; 2) to increase the solubility of the recombinant protein; and 3) to aid in the purification of the recombinant protein by acting as a ligand in affinity purification 4) to provide a “tag” for later detection of the protein. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase.

Typical fusion expression vectors include pGEX (Pharmacia Biotech Inc; Smith, D. B. and Johnson, K. S. (1988) Gene 67: 31-40), pMAL (New England Biolabs, Beverly, Mass.) and pRIT5 (Pharmacia, Piscataway, N.J.) which fuse glutathione S-transferase (GST), maltose E binding protein, or protein A, respectively.

Examples of suitable inducible non-fusion E. coli expression vectors include pTrc (Amann et al, (1988) Gene 69: 301-315), pLG338, pACYC184, pBR322, pUC18, pUC19, pKC30, pRep4, pHS1, pHS2, pPLc236, pMBL24, pLG200, pUR290, pIN-IIII 13-B1, egt11, pBdCl, and pET 11d (Studier et al., Gene Expression Technology: Methods in Enzymology 185, Academic Press, San Diego, Calif. (1990) 60-89; and Pouwels et al., eds. (1985) Cloning Vectors. Elsevier: New York IBSN 0 444 904018). Target gene expression from the pTrc vector relies on host RNA polymerase transcription from a hybrid tip-lac fusion promoter, Target gene expression from the pET lid vector relies on transcription from a T7 gnlO-lac fusion promoter mediated by a coexpressed viral RNA polymerase (T7gnl). This viral polymerase is supplied by host strains BL21 (DE3) or HMS174 (DE3) from a resident X prophage harboring a T7gnl gene under the transcriptional control of the lac.UV 5 promoter. For transformation of other varieties of bacteria, appropriate vectors may be selected. For example, the plasmids pIJ101, pIJ364, pIJ702 and pIJ361 are known to be useful in transforming Streptomyces, while plasmids pUB110, pC194 or pBD214 are suited for transformation of Bacillus species. Several plasmids of use in the transfer of genetic information into Corynebacterium include pHM1519, pBL1, pSA77 or pAJ667 (Pouwels et al., eds. (1985) Cloning Vectors. Elsevier: New York IBSN 0 444 904018).

Examples of suitable C. glutamicum and E. coli shuttle vectors are e.g. pClik5aMCS (WO2005059093) or can be found in Eikmanns et al (Gene. (1991) 102, 93-8).

Examples for suitable vectors to manipulate Corynebacteria can be found in the Handbook of Corynebacterium (edited by Eggeling and Bott, ISBN 0-8493-1821-1, 2005). One can find a list of E. coli-C. glutamicum shuttle vectors (table 23.1), a list of E. coli-C. glutamicum shuttle expression vectors (Table 23.2), a list of vectors which can be used for the integration of DNA into the C. glutamicum chromosome (Table 23.3), a list of expression vectors for integration into the C. glutamicum chromosome (Table 23.4.) as well as a list of vectors for site-specific Integration into the C. glutamicum chromosome (Table 23.6).

In another embodiment, the protein expression vector is a yeast expression vector. Examples of vectors for expression in yeast S. cerevisiae include pYepSec1 (Baldari, et al., (1987) Embo J. 6: 229-234), 2i, pAG-1, Yep6, Yep13, pEMBLYe23, pMFa (Kurjan and Herskowitz, (1982) Cell 30: 933-943), pJRY88 (Schultz et al, (1987) Gene 54: 113-123), and pYES2 (Invitrogen Corporation, San Diego, Calif.). Vectors and methods for the construction of vectors appropriate for use in other fungi, such as the filamentous fungi, include those detailed in: van den Hondel, C. A. M. J. J. & Punt, P. J. (1991) in: Applied Molecular Genetics of Fungi, J. F. Peberdy, et al., eds., p. 1-28, Cambridge University Press: Cambridge, and Pouwels et al., eds. (1985) Cloning Vectors. Elsevier: New York (IBSN 0 444 904018).

For the purposes of the present invention, an operative link is understood to be the sequential arrangement of promoter, coding sequence, terminator and, optionally, further regulatory elements in such away that each of the regulatory elements can fulfill its function, according to its determination, when expressing the coding sequence.

In another embodiment, the modified nucleotide sequences as mentioned above may be expressed in unicellular plant cells (such as algae) or in plant cells from higher plants (e.g., the spermatophytes, such as crop plants). Examples of plant expression vectors include those detailed in: Becker, D., Kemper, E., Schell, J. and Masterson, R. (1992) Plant Mol. Biol. 20: 1195-1197; and Bevan, M. W. (1984) Nucl. Acid. Res. 12: 8711-8721, and include pLGV23, pGHlac+, pBIN19, pAK2004, and pDH51 (Pouwels et al., eds. (1985) Cloning Vectors. Elsevier: New York IBSN 0 444 904018).

For other suitable expression systems for both prokaryotic and eukaryotic cells see chapters 16 and 17 of Sambrook, J. et al. Molecular Cloning: A Laboratory Manual. 3rd ed., Cold Spring Harbor Laboratory, Cold Spring Harbor Laboratory Press, Cold Spring Harbor, N.Y., 2003.

In another embodiment, the recombinant mammalian expression vector is capable of directing expression of the nucleic acid preferentially in a particular cell type, e.g. in plant cells (e.g., tissue-specific regulatory elements are used to express the nucleic acid). Tissue-specific regulatory elements axe known in the art.

Another aspect of the invention pertains to organisms or host cells into which a recombinant expression vector of the invention has been introduced. The terms “host cell” and “recombinant host cell” are used interchangeably herein. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein.

Vector DNA can be introduced into prokaryotic or eukaryotic cells via conventional transformation or transfection techniques. As used herein, the terms “transformation” and “transfection”, “conjugation” and “transduction” are intended to refer to a variety of art-recognized techniques for introducing foreign nucleic acid (e.g., linear DNA or RNA (e.g., a linearized vector or a gene construct alone without a vector) or nucleic acid in the form of a vector (e.g., a plasmid, phage, phasmid, phagemid, transposon or other DNA) into a host cell., including calcium phosphate or calcium chloride co-precipitation, DEAE-dextran-mediated transfection, lipofection, natural competence, conjugation chemical-mediated transfer, or electroporation. Suitable methods for transforming or transfecting host cells can be found in Sambrook, et al. (Molecular Cloning: A Laboratory Manual. 3rd ed., Cold Spring Harbor Laboratory, Cold Spring Harbor Laboratory Press, Cold Spring Harbor, N.Y., 2003), and other laboratory manuals.

In order to identify and select these integrants, a gene that encodes a selectable marker (e.g., resistance to antibiotics) is generally introduced into the host cells along with the gene of interest. Preferred selectable markers include those which confer resistance to drugs, such as G418, hygromycin, kanamycine, tratracycleine, ampicillin and methotrexate. Nucleic acid encoding a selectable marker can be introduced into a host cell on the same vector as that encoding the above-mentioned modified nucleotide sequences or can be introduced on a separate vector. Cells stably transfected with the introduced nucleic acid can be identified by drug selection (e.g., cells that have incorporated the selectable marker gene will survive, while the other cells die).

When plasmids without an origin of replication and two different marker genes are used (e.g. pClik int sacB), it is also possible to generate marker-free strains which have part of the insert inserted into the genome. This is achieved by two consecutive events of homologous recombination (see also Becker et al., APPLIED AND ENVIRONMENTAL MICROBIOLOGY, 71 (12), p. 8587-8596). The sequence of plasmid pClik int sacB can be found in WO20G5059G93; SEQ ID 24; the plasmid is called pCIS in this document).

In another embodiment, recombinant microorganisms can be produced which contain selected systems which allow for regulated expression of the introduced gene. For example, inclusion of one of the above-mentioned optimized nucleotide sequences on a vector placing it under control of the lac operon permits expression of the gene only in the presence of IPTG. Such regulatory systems are well known in the art.

In one embodiment, the method comprises culturing the organisms of invention (into which a recombinant expression vector or into which genome has been introduced a gene comprising the modified nucleotide sequences as mentioned above) in a suitable medium for fine chemical production. In another embodiment, the method further comprises isolating the fine chemical from the medium or the host cell.

Growth of Escherichia coli and Corynebacterium glutamicum-Media and Culture Conditions

The person skilled in the art is familiar with the cultivation of common microorganisms such as C. glutamicum and E. coli. Thus, a general teaching will be given below as to the cultivation of C. glutamicum. Corresponding information may be retrieved from standard textbooks for cultivation of E. coli.

E. coli strains are routinely grown in MB and LB broth, respectively (Follettie et al. (1993) J. Bacteriol. 175, 4096-4103). Minimal media for E. coli is M9 and modified MCGC (Yoshihama et al. (1985) J. Bacterial. 162, 591-507), respectively. Glucose may be added at a final concentration of 1%. If appropriate, antibiotics may be added in the following amounts (micrograms per millilitre): ampicillin, 50; kanamycin, 25; nalidixic acid, 25, Amino acids, vitamins, and other supplements may be added in the following amounts: methionine, 9.3 mM; arginine, 9.3 mM; histidine, 9.3 mM; thiamine, 0.05 mM. E. coli cells are routinely grown at 37 C, respectively.

Genetically modified Corynebacteria are typically cultured in synthetic or natural growth media. A number of different growth media for Corynebacteria are both well-known and readily available (Liebl et al. (1989) Appl. Microbiol. Biotechnol., 32: 205-210; von der Osten et al. (1998) Biotechnology Letters, 11: 11-16; Patent DE 4,120,867; Liebl (1992) “The Genus Corynebacterium, in: The Procaryotes, Volume II, Balows, A. et al., eds. Springer-Verlag) or Handbook of Corynebacterium glutamicum (2005) ISBN 0-8493-1821-1).

These media consist of one or more carbon sources, nitrogen sources, inorganic salts, vitamins and trace elements. Preferred carbon sources are sugars, such as mono-, di-, or polysaccharides. For example, glucose, fructose, mannose, galactose, ribose, sorbose, ribose, lactose, maltose, sucrose, glycerol, raffinose, starch or cellulose serve as very good carbon sources.

It is also possible to supply sugar to the media via complex compounds such as molasses or other by-products from sugar refinement. It can also be advantageous to supply mixtures of different carbon sources. Other possible carbon sources are alcohols and organic acids, such as methanol, ethanol, acetic acid or lactic acid. Nitrogen sources are usually organic or inorganic nitrogen compounds, or materials which contain these compounds. Exemplary nitrogen sources include ammonia gas or ammonia salts, such as NH₄Cl or (NH₄)₂S0₄, NH₄0H, nitrates, urea, amino acids or complex nitrogen sources like corn steep liquor, soy bean flour, soy bean protein, yeast extract, meat extract and others.

The overproduction of methionine is possible using different sulfur sources. Sulfates, thiosulfates, sulfites and also more reduced sulfur sources like H₂S and sulfides and derivatives can be used. Also organic sulfur sources like methyl mercaptan, thioglycolates, thiocyanates, and thiourea, sulfur containing amino acids like cysteine and other sulfur containing compounds can be used, to achieve efficient methionine production. Formate may also be possible as a supplement as are other Cl sources such as methanol or formaldehyde.

Inorganic salt compounds which may be included in the media include the chloride-, phosphorous- or su (fate-salts of calcium, magnesium, sodium, cobalt, molybdenum, potassium, manganese, zinc, copper and iron, Chelating compounds can be added to the medium to keep the metal ions in solution. Particularly useful chelating compounds include dihydroxyphenols, like catechol or protocatechuate, or organic acids, such as citric acid. It is typical for the media to also contain other growth factors, such as vitamins or growth promoters, examples of which include biotin, riboflavin, thiamine, folic acid, nicotinic acid, pantothenate and pyridoxine. Growth factors and salts frequently originate from complex media components such as yeast extract, molasses, corn steep liquor and others. The exact composition of the media compounds depends strongly on the immediate experiment and is individually decided for each specific case. Information about media optimization is available in the textbook “Applied Microbiol. Physiology, A Practical Approach (Eds. P. M. Rhodes, P. P. Stanbury, IRL Press (1997) pp. 53-73, ISBN 0 19 963577 3). It is also possible to select growth media from commercial suppliers, like standard 1 (Merck) or BHI (grain heart infusion, DIFCO) or others.

All medium components should be sterilized, either by heat (20 minutes at 1.5 bar and 121 C) or by sterile filtration. The components can either be sterilized together or, if necessary, separately.

All media components may be present at the beginning of growth, or they can optionally be added continuously or batch wise. Culture conditions are defined separately for each experiment.

The temperature should be in a range between 15° C. and 45° C. The temperature can be kept constant or can be altered during the experiment The pH of the medium may be in the range of 5 to 8.5, preferably around 7.0, and can be maintained by the addition of buffers to the media. An exemplary buffer for this purpose is a potassium phosphate buffer. Synthetic buffers such as MOPS, HEPES, ACES and others can alternatively or simultaneously be used. It is also possible to maintain a constant culture pH through the addition of NaOH or NH₄OH during growth. If complex medium components such as yeast extract are utilized, the necessity for additional buffers may be reduced, due to the fact that many complex compounds have high buffer capacities, if a fermentor is utilized for culturing the microorganisms, the pH can also be controlled using gaseous ammonia.

The incubation time is usually in a range from several hours to several days. This time is selected in order to permit the maximal amount of product to accumulate in the broth. The disclosed growth experiments can be carried out in a variety of vessels, such as microtiter plates, glass tubes, glass flasks or glass or metal fermentors of different sizes. For screening a large number of clones, the microorganisms should be cultured in microtiter plates, glass tubes or shake flasks, either with or without battles. Preferably 100 ml shake flasks are used, filled with 10% (by volume) of the required growth medium. The flasks should be shaken on a rotary shaker (amplitude 25 mm) using a speed-range of 100-300′rpm. Evaporation losses can be diminished by the maintenance of a humid atmosphere: alternatively, a mathematical correction for evaporation losses should be performed.

If genetically modified clones are tested, an unmodified control clone or a control clone containing the basic plasmid without any insert should also be tested. The medium is inoculated to an OD600 of 0.5-1.5 using cells grown on agar plates, such as CM plates (10 g/l glucose, 2.5 g/l NaCl, 2 g/l urea, 10 g/l polypeptone, 5 g/l yeast extract, 5 g/l meat extract. 22 g/l NaCl, 2 g/l urea, 10 g/l polypeptone, 5 g/l yeast extract, 5 g/l meat extract, 22 g/l agar, pH 6.8 with 2M NaOH) that had been incubated at 30° C. Inoculation of the media is accomplished by either introduction of a saline suspension of C. glutamicum cells from CM plates or addition of a liquid preculture of this bacterium.

### Definition of Recombination Protocol

In the following it will be described how a strain of C. glutamicum with increased efficiency of methionine production can be constructed implementing the findings of the above predictions. Before the construction of the strain is described, a definition of a recombination event/protocol is given that will be used in the following.

“Campbell in,” as used herein, refers to a transformant of an original host cell in which an entire circular double stranded DNA molecule (for example a plasmid being based on pCLIK int sacB has integrated into a chromosome by a single homologous recombination event (a cross-in event), and that effectively results in the insertion of a linearized version of said circular DNA molecule into a first DNA sequence of the chromosome that is homologous to a first DNA sequence of the said circular DNA molecule. “Campbelled in” refers to the linearized DNA sequence that has been integrated into the chromosome of a “Campbell in” transformant. A “Campbell in” contains a duplication of the first homologous DNA sequence, each copy of which includes and surrounds a copy of the homologous recombination crossover point. The name comes from Professor Alan Campbell, who first proposed this kind of recombination.

“Campbell out,” as used herein, refers to a cell descending from a “Campbell in” transformant, in which: a second homologous recombination event (a cross out event) has occurred between a second DNA sequence that is contained on the linearized inserted DNA of the “Campbelled in” DNA, and a second DNA sequence of chromosomal origin, which is homologous to the second DNA sequence of said linearized insert, the second recombination event resulting in the deletion (jettisoning) of a portion of the integrated DNA sequence, but, importantly, also resulting in a portion (this can be as little as a single base) of the integrated Campbelled in DNA remaining in the chromosome, such that compared to the original host cell, the “Campbell out” cell contains one or more intentional changes in the chromosome (for example, a single base substitution, multiple base substitutions, insertion of a heterologous gene or DNA sequence, insertion of an additional copy or copies of a homologous gene or a modified homologous gene, or insertion of a DNA sequence comprising more than one of these aforementioned examples listed above).

A “Campbell out” cell or strain is usually, but not necessarily, obtained by a counter-selection against a gene that is contained in a portion (the portion that is desired to be jettisoned) of the “Campbelled in” DNA sequence, for example the Bacillus subtilis sacB gene, which is lethal when expressed in a cell that is grown in the presence of about 5% to 10% sucrose. Either with or without a counter-selection, a desired “Campbell out” cell can be obtained or identified by screening for the desired cell, using any screenable phenotype, such as, but not limited to, colony morphology, colony color, presence or absence of antibiotic resistance, presence or absence of a given DNA sequence by polymerase chain reaction, presence or absence of an auxotrophy, presence or absence of an enzyme, colony nucleic acid hybridization, antibody screening, etc. The term “Campbell in” and “Campbell out” can also be used as verbs in various tenses to refer to the method or process described above.

It is understood that the homologous recombination events that leads to a “Campbell in” or “Campbell out” can occur over a range of DNA bases within the homologous DNA sequence, and since the homologous sequences will be identical to each other for at least part of this range, it is not usually possible to specify exactly where the crossover event occurred. In other words, it is not possible to specify precisely which sequence was originally from the inserted DNA, and which was originally from the chromosomal DNA. Moreover, the first homologous DNA sequence and the second homologous DNA sequence are usually separated by a region of partial non-homology, and it is this region of non-homology that remains deposited in a chromosome of the “Campbell out” cell.

For practicality, in C. glutamicum, typical first and second homologous DNA sequence are at least about 200 base pairs in length, and can be up to several thousand base pairs in length, however, the procedure can be made to work with shorter or longer sequences. For example, a length for the first and second homologous sequences can range from about 500 to 2000 bases, and the obtaining of a “Campbell out” from a “Campbell in” is facilitated by arranging the first and second homologous sequences to be approximately the same length, preferably with a difference of less than 200 base pairs and most preferably with the shorter of the two being at least 70% of the length of the longer in base pairs. The “Campbell In and -Out-method” is described in WO2007012078

The invention will now be illustrated by means of various examples. These examples are however in no way meant to limit the invention in any way.

### EXAMPLES

In the following it will be shown how the codon usage of abundant proteins in C. glutamicum was identified. Furthermore, examples are presented which show that usage of modified nucleotide sequences which have been optimized with regard to either the codon usage of abundant proteins or the organism of C. glutamicum can be used to increase the amount of a protein in C. glutamicum. This is shown for foreign genes as well as endogenous genes.

1. Identification of Abundant Proteins in C. glutamicum

Cellular extracts were prepared from the C. glutamicum strain ATCC13032 and of some derivatives. For this purpose, 250 mg of cell grown under standard conditions were pelleted and suspended in 750 μl lysis buffer (20 mM TRIS, 5 mM EDTA, pH 7.5) containing a protease inhibitor mix (Complete, Roche). Cell disruption was carried out at 4° C. in a mixer mill (Retsch, M M 2000) using 0.25-0.5 mm glass beads. Cell debris was removed by centrifugation at 22.000 rpm for 1 hour at 4° C. Protein concentrations were determined by the Popov (Popove et al. (1975) Acta. Biol. Med. Germ, 34, 1441-1446). Cell extracts were used immediately or frozen in aliquots at −80° C.

For 2D polyacrylamide gel electrophoresis of proteins 30 μg of crude protein extract was resuspended 450 μl of rehydration buffer (8M urea, 2M thiourea, 1% CHAPS, 20 mM DTT, 1% Ampholines 3.5-10) and a few grains of bromophenol blue. For isoelectric focussing precast 24 cm-IPG strips with a linear pH gradient of 4.5 to 5.5 were used in a Multiphor II isoelectric focussing unit (Amersham Biosciences). Proteins were focused using a gradient programme up to 3500 V resulting 65.000 Vh in total. Focused IPG gels were equilibrated twice for 15 minutes in a buffer containing 1.5 M Tris-HCl (pH 8.8), 6M urea, 30% (vol/vol) glycerol, 2% (wt/vol) sodium dodecyl sulfate, and 1% (wt/vol) DTT. For the second equilibration step DDT was replaced by 5% (wt/vol) iodoacetamide, and a few grains of bromophenol blue were added. The second dimension was run in sodium dodecyl sulfate-12.5% polyacrylamide gels in an Ettan Dalt apparatus (Amersham Biosciences) as recommended by the manufacturer, and gels were subsequently silver stained (Blum et al. (1987), Electrophoresis, 8, 93-99) in a home made staining automat.

Protein spots were excised from preparative Coomassie-stained gels (300 μg total protein load each) and digested with modified trypsin (Roche, Mannheim) as described by Hermann et al. (Electrophoresis (2001), 22, 1712-1723). Mass spectrometrical identifications were performed on an LCQ advantage (Thermo Electron) after nano-HPLC separation of the peptides (LC Packings, RP18 column, length 15 cm, i.d. 75 μm), using the MASCOT software (David et at. (1999) Electrophoresis, 20, 3551-3567).

Based on the 2D gel electrophoresis results of different gels 14 proteins were identified as being abundant in C. glutamicum as these proteins could be observed at high amounts in ail gels. These proteins are: Elongation Factor Tu (Genbank accession no: X77034), glycerine-aldehyde-3-phosphate-dehydrogenase (Genbank accession no: BX927152, ±, nt. 289401-288397), fructose bisphosphate aldolase (Genbank accession no: BX927156, ±, nt. 134992-133958). Elongation Factor Ts (Genbank accession no: BX927154, ±, nt. 14902-14075), hypothetical protein (Genbank accession no: BX927155, ±, nt. 213489-214325), enolase (Genbank accession no: BX927150, nt. 338561-339838) peptidyl-prolyl cis-trans isomerase (Genbank accession no: BX927148, nt. 34330-34902), superoxide dismutase (Genbank accession no: AB055218) phospho-glycerate dehydrogenase (Genbank accession no: BX927T51, nt. 306039-307631) SSU Rib protein SIP (Genbank accession no: BX927152, ±, nt. 26874-28334) triosephosphate-isomerase (Genbank accession no: BX927152, ±, nt. 286884-286105) isopropyl malate synthase (Genbank accession no: X70959) butan-2,3-dioldehydrogenase (Genbank accession no: BX927156, nt. 20798-21574) and fumarat hydratase (Genbank accession no: BX927151, ±, nt. 18803-17394).

The coding sequences of these genes were then fed into the “Cusp” function of the EMBOSS tool box using standard parameters in an independent approach the genomic sequence of the complete C. glutamicum strain ATCC13032 was used to generate a codon usage table for the organism as a whole.

The codon usage frequencies as determined for the aforementioned 14 abundant proteins were used to calculate codon usage frequencies for abundant proteins in C. glutamicum. The codon relative codon usage frequencies of abundant proteins in C. glutamicum are found in Table 2, while the relative codon usage frequencies of the organism as a whole are found in Table 1.

TABLE 1
Relative codon usage frequencies of Corynebacterium glutamicum
ATCC 13032.

| | | | | | | | |
|---|---|---|---|---|---|---|---|
| UUU | 37.1 | UCU | 17.3 | UAU | 33.8 | UGU | 36.5 |
| UUC | 62.9 | UCC | 33.6 | UAC | 66.2 | UGC | 63.5 |
| UUA | 5.3 | UCA | 13.0 | UAA | 53.1 | UGA | 16.7 |
| UUG | 20.3 | UCG | 11.9 | UAG | 30.2 | UGG | 100 |
| CUU | 17.2 | CCU | 23.3 | CAU | 32.1 | CGU | 24.5 |
| CUC | 22.5 | CCC | 20.2 | CAC | 67.9 | CGC | 44.7 |
| CUA | 6.1 | CCA | 34.9 | CAA | 38.5 | CGA | 11.8 |
| CUG | 28.6 | CCG | 21.6 | CAG | 61.5 | CGG | 8.8 |
| AUU | 37.7 | ACU | 20.4 | AAU | 33.4 | AGU | 7.8 |
| AUC | 59.2 | ACC | 52.9 | AAC | 66.4 | AGC | 16.4 |
| AUA | 3.1 | ACA | 12.5 | AAA | 39.9 | AGA | 4.1 |
| AUG | 100 | ACG | 14.2 | AAG | 60.1 | AGG | 6.1 |
| GUU | 26.0 | GCU | 23.7 | GAU | 55.6 | GGU | 30.3 |
| GUC | 27.7 | GCC | 25.4 | GAC | 44.4 | GGC | 42.4 |
| GUA | 10.1 | GCA | 29.3 | GAA | 56.3 | GGA | 18.9 |
| GUG | 36.2 | GCG | 21.6 | GAG | 43.7 | GGG | 8.4 |
| ATG* | 72.5 | | | | | | |
| GTG* | 20.5 | | | | | | |
| TTG* | 7.0 | | | | | | |

*designates start codons; relative Frequencies are in percentage.

TABLE 2
Relative codon usage frequencies of 14 abundant proteins in
Corynebacterium glutamicum ATCC 13032.

| | | | | | | | |
|---|---|---|---|---|---|---|---|
| UUU | 10.6 | UCU | 20.2 | UAU | 3.6 | UGU | 25.0 |
| UUC | 89.4 | UCC | 65.4 | UAC | 96.4 | UGC | 75.0 |
| UUA | 0.8 | UCA | 3.3 | UAA | 92.9 | UGA | 0.0 |
| UUG | 7.5 | UCG | 2.2 | UAG | 7.1 | UGG | 100 |
| CUU | 20.8 | CCU | 37.6 | CAU | 5.8 | CGU | 39.6 |
| CUC | 25.4 | CCC | 4.4 | CAC | 94.2 | CGC | 57.6 |
| CUA | 2.6 | CCA | 51.4 | CAA | 7.7 | CGA | 2.2 |
| CUG | 42.9 | CCG | 6.6 | CAG | 92.3 | CGG | 0.6 |
| AUU | 17.1 | ACU | 18.9 | AAU | 9.7 | AGU | 0.8 |
| AUC | 82.6 | ACC | 78.9 | AAC | 90.3 | AGC | 8.1 |
| AUA | 0.3 | ACA | 1.4 | AAA | 7.1 | AGA | 0.0 |
| AUG | 100 | ACG | 0.8 | AAG | 92.9 | AGG | 0.0 |
| GUU | 47.9 | GCU | 46.8 | GAU | 34.9 | GGU | 32.3 |
| GUC | 34.0 | GCC | 9.9 | GAC | 65.1 | GGC | 59.0 |
| GUA | 6.5 | GCA | 35.9 | GAA | 32.5 | GGA | 8.2 |
| GUG | 11.6 | GCG | 7.4 | GAG | 67.5 | GGG | 0.5 |
| ATG* | 78.6 | | | | | | |
| GTG* | 21.4 | | | | | | |
| TTG* | 0.0 | | | | | | |

*indicates start codons; relative Frequencies are in percentage.

Table 2 was then used to determine the codons that are used most frequently for each amino acid in the abundant proteins of C. glutamicum. This information is displayed in Table 3 below.

TABLE 3
Codon usage of 14 abundant proteins in Corynebacterium glutamicum
ATCC 13032.

| | |
|---|---|
| UUC | F |
| UCC | S |
| UAC | Y |
| UGC | C |
| UAA | Stop |
| ATG if start codon | M |
| UGG | W |
| CUG | L |
| CCA | P |
| CAC | H |
| CGC | R |
| CAG | Q |
| AUC | I |
| ACC | T |
| AAC | N |
| AUG | M |
| AAG | K |
| GUU | V |
| GCU | A |
| GAC | D |
| GGC | G |
| GAG | E |

Table 4 shows the frequencies of codons which are not calculated on the basis of codons encoding a specific amino acid, but on the basis of all codons for all amino acids. The values in brackets indicate the absolute number of the respective codon. The relative frequencies of Table 1 were calculated on the basis of these absolute numbers. The values refer to the organism of C. glutamicum.

TABLE 4
Codon usage of Corynebacterium glutamicum ATCC 13032.

| | | | | | | | |
|---|---|---|---|---|---|---|---|
| UUU | 13.4(25821) | UCU | 11.0(21227) | UAU | 7.5(14384) | UGU | 2.4(4605) |
| UUC | 22.8(43837) | UCC | 21.4(41118) | UAC | 14.7(28214) | UGC | 4.2(8015) |
| UUA | 5.1(9795) | UCA | 8.3(15898) | UAA | 1.7(3272) | UGA | 0.5(1032) |
| UUG | 19.6(37762) | UCG | 7.6(14639) | UAG | 1.0(1859) | UGG | 14.1(27072) |
| CUU | 16.7(32074) | CCU | 11.3(21668) | CAU | 6.8(12991) | CGU | 13.7(26310) |
| CUC | 21.8(41988) | CCC | 9.7(18716) | CAC | 14.3(27445) | CGC | 24.9(47939) |
| CUA | 5.9(11320) | CCA | 16.9(32429) | CAA | 13.0(24975) | CGA | 6.6(12698) |
| CUG | 27.7(53261) | CCG | 10.4(20070) | CAG | 20.7(39864) | CGG | 4.9(9466) |
| AUU | 21.7(41804) | ACU | 12.6(24184) | AAU | 10.9(21056) | AGU | 4.9(9515) |
| AUC | 34.1(65557) | ACC | 32.5(62592) | AAC | 21.8(42037) | AGC | 10.4(20019) |
| AUA | 1.8(3483) | ACA | 7.7(14747) | AAA | 13.9(26703) | AGA | 2.3(4445) |
| AUG | 22.1(42484) | ACG | 8.8(16879) | AAG | 20.9(40213) | AGG | 3.3(6398) |
| GUU | 20.8(40069) | GCU | 25.4(48864) | GAU | 33.0(63429) | GGU | 24.3(46678) |
| GUC | 22.2(42696) | GCC | 27.2(52264) | GAC | 26.4(50716) | GGC | 34.0(65427) |
| GUA | 8.1(15628) | GCA | 31.3(60329) | GAA | 35.7(68737) | GGA | 15.2(29219) |
| GUG | 28.9(55708) | GCG | 23.2(44613) | GAG | 27.7(53381) | GGG | 6.7(12923) |

Frequencies are indicated after the codons in/1000.

Table 5 shows the frequencies of codons which were not calculated on the basis of codons encoding a specific amino acid, but on the basis of all codons for all amino acids. The values in brackets indicate the absolute number of the respective codon. The relative frequencies of Table 2 were calculated on the basis of these absolute numbers. The values refer to the group of abundant proteins in C. glutamicum.

TABLE 5
Codon usage of 14 abundant proteins in Corynebacterium glutamicum ATCC
13032.

| | | | | | | | |
|---|---|---|---|---|---|---|---|
| UUU | 3.6(18) | UCU | 10.9(55) | UAU | 0.8(4) | UGU | 1.2(6) |
| UUC | 30.0(152) | UCC | 35.2(178) | UAC | 21.1(107) | UGC | 3.6(18) |
| UUA | 0.6(3) | UCA | 1.8(9) | UAA | 2.6(13) | UGA | 0.0(0) |
| UUG | 5.7(29) | UCG | 1.2(6) | UAG | 0.2(1) | UGG | 8.3(42) |
| CUU | 16.0(81) | CCU | 13.4(68) | CAU | 1.2(6) | CGU | 17.4(88) |
| CUC | 19.6(99) | CCC | 1.6(8) | CAC | 19.4(98) | CGC | 25.3(128) |
| CUA | 2.0(10) | CCA | 18.4(93) | CAA | 2.6(13) | CGA | 1.0(5) |
| CUG | 33.0(167) | CCG | 2.4(12) | CAG | 30.6(155) | CGG | 0.2(1) |
| AUU | 9.9(50) | ACU | 10.5(53) | AAU | 4.0(20) | AGU | 0.4(2) |
| AUC | 47.8(242) | ACC | 43.7(221) | AAC | 36.8(186) | AGC | 4.3(22) |
| AUA | 0.2(1) | ACA | 0.8(4) | AAA | 3.6(18) | AGA | 0.0(0) |
| AUG | 17.2(87) | ACG | 0.4(2) | AAG | 46.4(235) | AGG | 0.0(0) |
| GUU | 43.5(220) | GCU | 54.9(278) | GAU | 21.9(111) | GGU | 28.1(142) |
| GUC | 30.8(156) | GCC | 11.7(59) | GAC | 40.9(207) | GGC | 51.2(259) |
| GUA | 5.9(30) | GCA | 42.1(213) | GAA | 27.9(141) | GGA | 7.1(36) |
| GUG | 10.5(53) | GCG | 8.7(44) | GAG | 57.9(293) | GGG | 0.4(2) |

Frequencies are indicated after the codons in/1000.

Surprisingly there are many significant differences in the codon usage between tables generated by using all proteins (whole genome, Table 1) compared to the situation where only the above-specified abundant genes are considered (Table 2). Some of the examples are shown in Table 6 below.

TABLE 6
Relative frequency of codons used

| Amino acid | Codon | Whole genome | Abundant proteins |
|---|---|---|---|
| q | caa | 38.5% | 7.7 |
| q | cag | 61.5% | 91.3 |
| y | tac | 66.0% | 96.4 |
| y | tat | 33.8% | 3.6 |

2. Improved Protein Expression of a Heterologous Gene in C. glutamicum

It was considered to use the above finding for optimizing gene expression of foreign heterologous genes in C. glutamicum.

To this end the coding sequence of lysine-2,3-aminomutase from Clostridium subterminale was used. The accession number for wild type aminomutase from C. subterminale is Q9XBQ8 (protein sequence), AF159146 (nucleotide sequence)

Introduction of the enzymatic activities of lysine-2,3 aminomutase in C. glutamicum is highly interesting because this enzyme catalyzes the isomerization of L-lysine into β-lysine. β-lysine as well as L-lysine may be interesting compounds as they can be used as precursor molecules in the production of ε-caprolactam which is used for industrially important polymers such as Nylon 6.

While L-lysine may also be used for ε-caprolactam synthetization via cyclization of L-lysine followed by deamination, β-lysine may be more interesting because deamination may be performed without the relatively expensive chemical hydroxylamine-Q-sulfonic acid.

Furthermore, β-lysine is also a constituent of antibiotics produced by Streptomyces and Norcardia such as viomycin, streptolin A, streptothricin, roseothricin, geomycin and myomicin. It may therefore be interesting to have an organism available that is derived from C. glutamicum and allows for efficient production of β-lysine by catalyzing the isomerization of naturally produced L-lysine.

However, as the gene for lysine 2,3-aminomutase is not present in C. glutamicum expression of the original C. subterminale sequence may not proceed efficiently enough.

For cloning of C. subterminale lysine 2,3-aminomutase the PGR primers WKJ90 (cctaacacagaaatgtc) (SEQ ID No. 3) and WKJ165 (cagtctgcatcgctaacatc) (SEQ ID No. 4) were used together with the chromosome of C. subterminale as a template to amplify a DNA fragment of up- and downstream regions including N- and C-terminal sequences of kamA gene respectively. The resulting amplification product was purified and subsequently the full sequence of the C. subterminale kamA gene which includes the gene for the aminomutase were amplified using PGR primers WKJ105 (ateticttggcagaacteatgggtaaaaaatcctttegta) (SEQ ID No. 5) and WKJ106 (gagagagatctagatagctgccaattattccggg) (SEQ ID No. 6). The amplified PGR fragment was purified, digested with restriction enzymes XhoI and MloI and ligated to the pClik5aMCS which had been digested with the same restriction enzymes.

As the codon usage for the C. subterminale kamA gene is quite different to that of the abundant, genes of C. glutamicum, expression of the C. subterminale kamA may not be efficient in a C. glutamicum lysine producing strain.

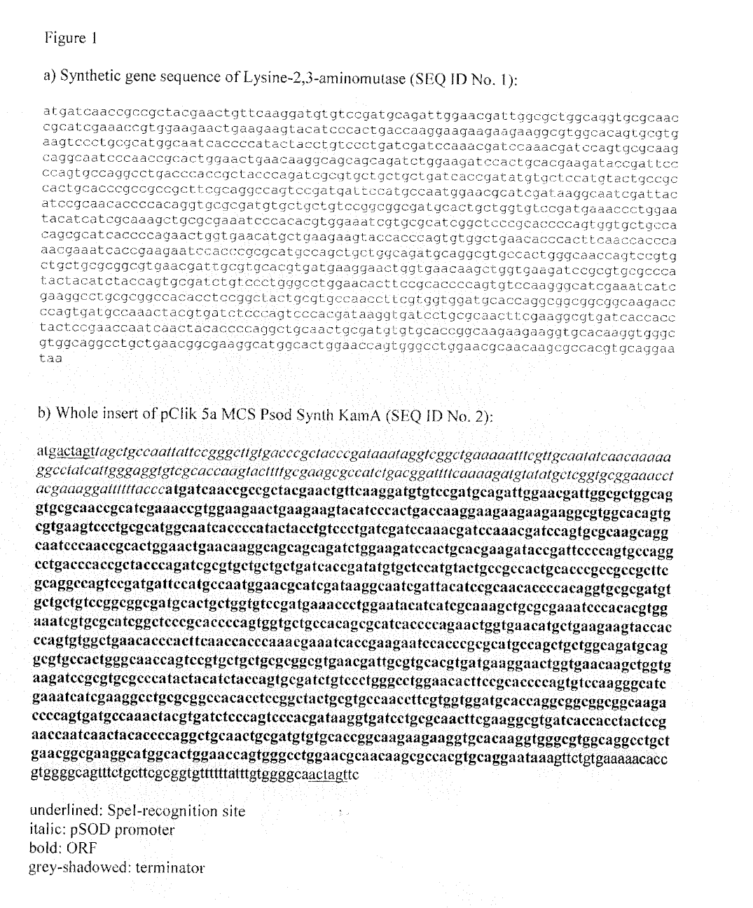

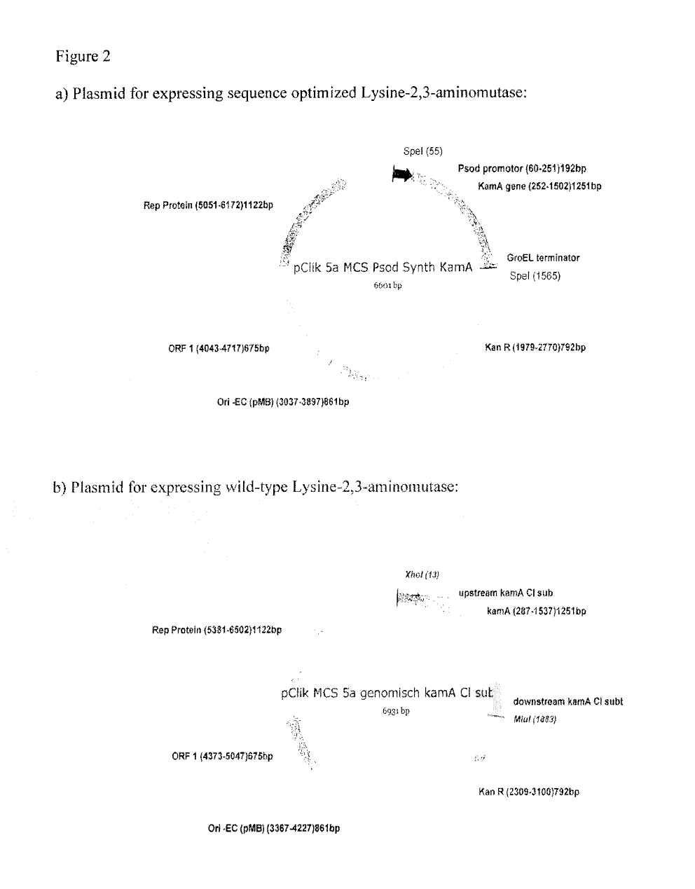

To enhance gene expression in C. glutamicum, a synthetic kamA gene was therefore created with the sequence of the synthetic gene being adapted to C. glutamicum codon usage on the basis of the codon usage as determined for the whole organism of C. glutamicum (SEQ ID No. 1). Furthermore, the synthetic kamA gene had a C. glutamicum sod A promoter (Psod) and a groEL terminator. The sequence of the synthetic kamA gene is shown in FIG. 1 (Seq ID No. 2). The genomic kamA gene was introduced into pClik using the endogenous kamA promoter (pClik 5a MCS genomisch kamA Cl sub, see FIG. 2b). The DNA constructs used for expression of the original sequence of C. subterminale aminomutase and the synthetic gene are schematically shown in FIG. 2

Subsequently, a lysine producing strain of C. glutamicum was transformed by electroporation with recombinant plasmids harboring the aforementioned synthetic lysine 2,3-aminomutase gene or the respective wild type C. subterminale lysine 2,3-aminomutase gene. The plasmids were based on pClik. Shaking flask experiments were performed on the recombinant strains to test β-lysine production. The same culture medium and conditions were employed.

For the control, the host strain and recombinant strain having the empty plasmid pClik5aMCS were tested in parallel. The strains were precultured on CM agar at 30° C. overnight. Cultured cells were then harvested in a microtube containing 1.5 ml of 0.9% NaCl and cell density was determined by the absorbance at 610 nm following vortex. For the main culture, suspended cells were inoculated to reach 1.5 of initial OD into 10 ml of the production medium contained in an autoclaved 100 ml of Erlenmeyer flask having 0.5 g of CaCO₃. In case a recombinant strain was cultured, 20 μg/ml of kanamycine was added to all media. Main culture was performed on a rotary shaker with 200 rpm at 30° C. for 48-78 hs.

For cell growth measurement, 0.1 ml of culture broth was mixed with 0.9 ml of 1N HCL to eliminate CaCO₃, and the absorbance at 610 nm was measured following appropriate dilution.

The concentration of β-lysine, lysine and residual sugar including glucose, fructose and sucrose were measured by HPLC method. Culture broth was centrifuged at 13,000 rpm for 5 min, diluted appropriately with water (if needed), filtrated with 0.22 μm filter, and followed by injection onto HPLC column.

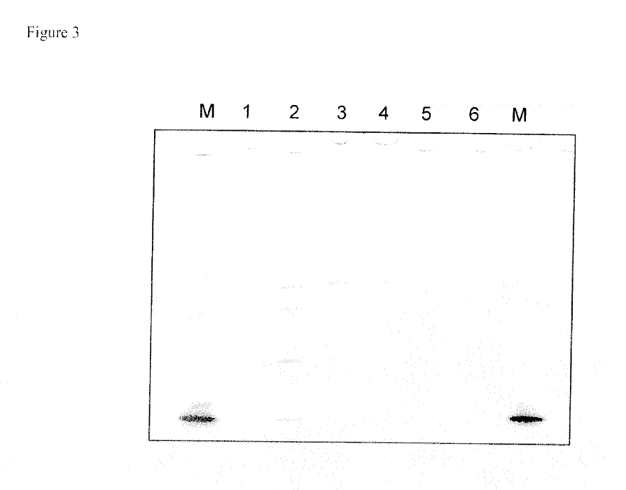

An accumulation of β-lysine was only observed in recombinant strains containing C. subterminale synthetic kamA gene. In addition, expression of the genes was confirmed by SDS-PAGE (see FIG. 3).

It has to be observed that, the two expression constructs may not been readily comparable. The synthetic gene was expressed under the control of the strong promoter Psod which, however, is not present in the construct containing the original sequence. However, it is assumed that the increased production of β-lysine would also be observed if the synthetic gene and the original constructs were expressed under the control of identical promoters.

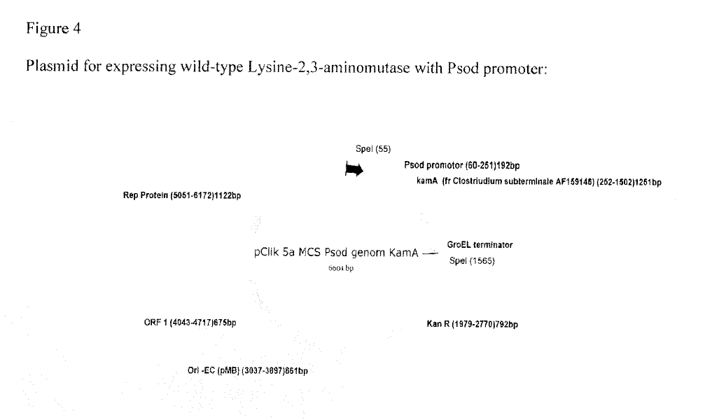

Accordingly a plasmid was constructed harbouring the genomic kamA gene under the control of a Psod promoter. Again the plasmid is based in pClik. A schematic representation of the resulting plasmid pClik 5a MCS Psod genom KamA (SEQ ID No. 7) is depicted in FIG. 4.

The pClik 5a MCS Psod genom KamA plasmid was expressed in C. glutamicum as described above.

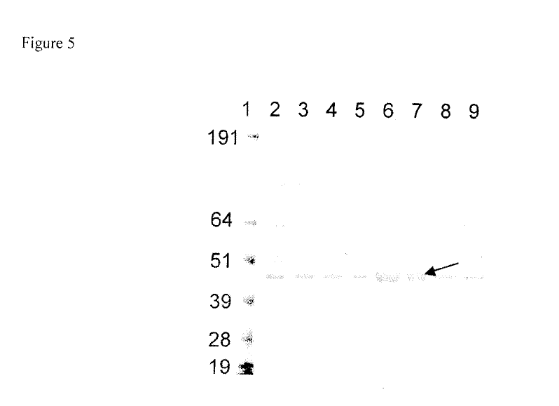

From two independent transformants, an overnight culture was grown and cell extracts were prepared. Equal amounts of total protein were loaded on an SDS-gel and the expression of the KamA protein was analyzed after Coomassie staining. As can be seen from FIG. 5, the codon optimized kamA gene under control of Psod results in a high level of KamA protein while the wild type kamA sequence, also controlled by the same promoter, does not give this high level of expression. The expected size of KamA which is 47 kDa by an arrow. Thus, the effect of increased protein level is due to the optimization of the codon usage.

3. Improved Protein Expression of lysA in C. glutamicum

In the following it is described how to increase the amount of lysA by adapting the codon usage as mentioned above.

3.1 Construction of Optimized lysA

The enzyme lysA is important for lysine biosynthesis. The codon usage of the coding sequence of lysA (Genbank accession no. 3344931) was determined using the Cusp function of the EMBOSS software package. The codon usage of the endogenous gene is depicted in table 7 below.

TABLE 7
codon usage of wild type lysA

| Codon | Amino acid | Fract | /1000 | Number |
|---|---|---|---|---|
| GCA | A | 0.448 | 58.427 | 26 |
| GCC | A | 0.328 | 42.697 | 19 |
| GCG | A | 0.069 | 8.989 | 4 |
| GCT | A | 0.155 | 20.225 | 9 |
| TGC | C | 0.750 | 6.742 | 3 |
| TGT | C | 0.250 | 2.247 | 1 |
| GAC | D | 0.800 | 53.933 | 24 |
| GAT | D | 0.200 | 13.483 | 6 |
| GAA | E | 0.824 | 62.921 | 28 |
| GAG | E | 0.176 | 13.483 | 6 |
| TTC | F | 1.000 | 35.955 | 16 |
| TTT | F | 0.000 | 0.000 | 0 |
| GGA | G | 0.195 | 17.978 | 8 |
| GGC | G | 0.561 | 51.685 | 23 |
| GGG | G | 0.049 | 4.494 | 2 |
| GGT | G | 0.195 | 17.978 | 8 |
| CAC | H | 1.000 | 26.966 | 12 |
| CAT | H | 0.000 | 0.000 | 0 |
| ATA | I | 0.000 | 0.000 | 0 |
| ATC | I | 0.773 | 38.202 | 17 |
| ATT | I | 0.227 | 11.236 | 5 |
| AAA | K | 0.545 | 13.483 | 6 |
| AAG | K | 0.455 | 11.236 | 5 |
| CTA | L | 0.073 | 6.742 | 3 |
| CTC | L | 0.220 | 20.225 | 9 |
| CTG | L | 0.512 | 47.191 | 21 |
| CTT | L | 0.098 | 8.989 | 4 |
| TTA | L | 0.000 | 0.000 | 0 |
| TTG | L | 0.098 | 8.989 | 4 |
| ATG | M | 1.000 | 13.483 | 6 |
| AAC | N | 0.750 | 26.966 | 12 |
| AAT | N | 0.250 | 8.989 | 4 |
| CCA | P | 0.529 | 20.225 | 9 |
| CCC | P | 0.294 | 11.236 | 5 |
| CCG | P | 0.000 | 0.000 | 0 |
| CCT | P | 0.176 | 6.742 | 3 |
| CAA | Q | 0.286 | 4.494 | 2 |
| CAG | Q | 0.714 | 11.236 | 5 |
| AGA | R | 0.000 | 0.000 | 0 |
| AGG | R | 0.000 | 0.000 | 0 |
| CGA | R | 0.000 | 0.000 | 0 |
| CGC | R | 0.783 | 40.449 | 18 |
| CGG | P | 0.043 | 2.247 | 1 |
| CGT | R | 0.174 | 8.989 | 4 |
| AGC | S | 0.250 | 15.730 | 7 |
| AGT | S | 0.000 | 0.000 | 0 |
| TCA | S | 0.071 | 4.494 | 2 |
| TCC | S | 0.607 | 38.202 | 17 |
| TCG | S | 0.000 | 0.000 | 0 |
| TCT | S | 0.071 | 4.494 | 2 |
| ACA | T | 0.091 | 4.494 | 2 |
| ACC | T | 0.818 | 40.449 | 18 |
| ACG | T | 0.045 | 2.247 | 1 |
| ACT | T | 0.045 | 2.247 | 1 |
| GTA | V | 0.195 | 17.978 | 3 |
| GTC | V | 0.220 | 20.225 | 9 |
| GTG | V | 0.341 | 31.461 | 14 |
| GTT | V | 0.244 | 22.472 | 10 |
| TGG | W | 1.000 | 4.494 | 2 |
| TAC | Y | 0.929 | 29.213 | 13 |
| TAT | Y | 0.071 | 2.247 | 1 |
| TAA | * | 0.000 | 0.000 | 0 |
| TAG | * | 0.000 | 0.000 | 0 |
| TGA | * | 0.000 | 0.000 | 0 |

Frequencies are indicated as /1000.

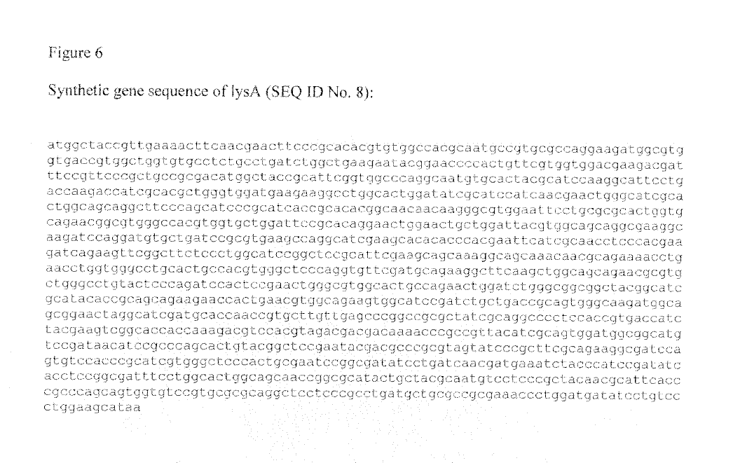

Starting from this endogenous sequence an optimized synthetic sequence was constructed. The synthetic optimized lysA sequence was provided by GeneArt GmbH (Regensburg, Germany). The sequence of the optimized lysA construct is depicted in FIG. 6 as Seq ID No. 8.

A cloning insert, to be cloned into pClik int sacB was obtained containing approximately 600 (593) nucleotides upstream of the coding region of lysA, the optimized lysA coding region and approximately 600 (606) nucleotide downstream of the coding region of lysA. This construct was obtained by a set of fusion PGR based which are outlined in table 9 below.

TABLE 8
Primer sequences for lysA

| | | | | | |
|---|---|---|---|---|---|
| | Sense | Antisense | | Amplified | |
| PCR | primer | primer | Template | region | Size [bp] |

| | | | | | |
|---|---|---|---|---|---|
| 1 | Old 540 | Old 541 | Genomic DNA | upstream lysA | 604 |
| | | | ATCC 13032 | | |
| 2 | Old 542 | Old 543 | Synthetic gene | cds lysA | 1377 |
| 3 | Old 544 | Old 545 | Genomic DNA | downstream | 618 |
| | | | ATCC 13032 | lysA | |
| 4 | Old 542 | Old 545 | PCR 2 and | Fusion cds | 1976 |
| | | | PCR 3 | and | |
| | | | | downstream | |
| 5 | Old 540 | Old 545 | PCR 1 and | Fusion | 2560 |
| | | | PCR 4 | upstream, cds | |
| | | | | and | |
| | | | | downstream | |

Old 540 ACTATGACGTCGGCGTTGAAGTCCTGATTGG (SEQ ID No. 12)
Old 541 TGTTACATCTTCTCCGGTGC (SEQ ID No. 13)
Old 542 GCACCGGAGAAGATGTAACAATGGCTACCGTTGAAAACTTCAA (SEQ ID No. 14)
Old 543 GGTCAGGCGTCGAAAAGCGTTATGCTTCCAGGGACAGGA (SEQ ID No. 15)
Old 544 CGCTTTTCGACGCCTGACC (SEQ ID No. 16)
Old 545 ACTACTTCTAGACGACAACTCCTACTACCTCTCC (SEQ ID No. 17)

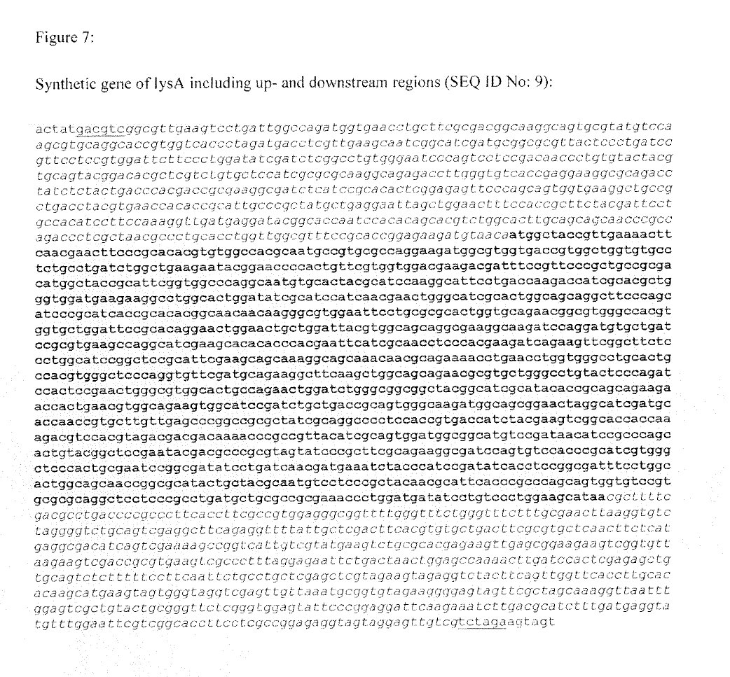

The sequence of the optimized sequence is shown in FIG. 6 (SEQ ID No. 8). The sequence of the complete cloning construct is shown in FIG. 7 as SEQ ID No. 9. Underlined are the Aat II and XbaI restriction sites which were introduced by the primers Old 540 and Old 545.

The PGR product was then purified, digested with Aat II and Xba I, purified again and ligated with pClik int sacB which had been linearized before with the same enzymes respectively. Integrity of the insert was confirmed by sequencing.

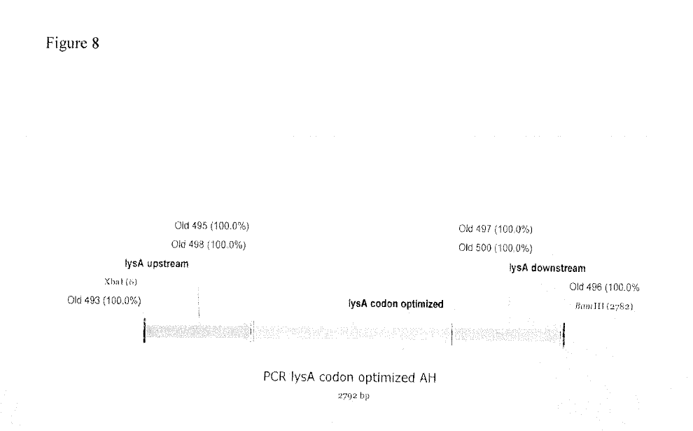

A general outline of the cloning construct is depicted in. FIG. 8.

3.3 Construction of C. glutamicum Strains Containing the Optimized Synthetic lysA Genes

The plasmid containing the optimized synthetic lysA gene can be used to replace the native coding region of the lysA gene by the coding region with the optimized coding usage. Two consecutive recombination events one in each of the up- and the downstream region respectively are necessary to change the complete coding sequence. The method of replacing the endogenous genes with the optimized genes is in principle described in the publication by Becker et al. (vide supra). The most important steps are:
Introduction of the plasmids in the strain by electroporation. The step is e.g. described in DE 10046870 which is incorporated by reference as far as introduction of plasmids into strains is disclosed therein.
Selection of clones that successfully have integrated the plasmid after a first homologous recombination event into the genome. This selection is achieved by growth on kanamycine-containing agar plates. In addition to that selection step, successful recombination can be checked via colony PGR.
By incubating a positive clone in a kanamycine-free medium a second recombination event is allowed for.
Clones in which the vector backbone are successfully removed by way of a second recombination event are then identified by growth on sucrose-containing medium. Only those clones will survive that have lost the vector backbone comprising the SacB gene.
Then, clones in which the two recombination events had led to replacement of the native lysA-coding region can be identified with PCR-specific primers.

For verification of successful integration of the synthetic lysA gene, a PGR analysis can be performed first. To this end the following primer pair can be used:

| | |
|---|---|
| Old 494: AACCGTGGAAAACTTCAAC | (SEQ ID No. 18) |
| Old 499: TCCAGGGACAGGATATCA | (SEQ ID No. 19) |

A PCR product of approximately 1327 bp in size is expected.

Successful manipulation can be further confirmed by Southern blotting:

Southern Blot lysA

Probes for Southern blotting can be made by PGR using the following oligonucleotides and pClik int sacB lysA codon optimized sequence as a template:

| | |
|---|---|
| Old494: AACCGTGGAAAACTTCAAC | (SEQ ID No. 18) |
| Old499: TTCCAGGGACAGGATATCA | (SEQ ID No, 19) |

Genomic DNA of the parent strain and the clones which are selected after PGR can be prepared, digested over night with an restriction enzyme as detailed below, separated on an 1% agarose gel and blotted onto a Nylon membrane according to standard methods. Detection can be done using a commercial Kit (Amersham) following the instructions of the manufacturer. For the following digest, one would expect the indicated fragments:

| | expected fragment size | expected fragment size |
|---|---|---|
| Enzyme | native lysA: | optimized lysA: |
| SalI/PstI | 1294 | 2806 |
| Bgl II/Mlu I | 3066, 4646 | 591, 363, 4284, 2475 |

The Southern Blot Analysis may be used to confirm the successful integration of the synthetic lysA gene.

As parent strains, C. glutamicum lysine producing strains can be used.

One may use different C. glutamicum strains for this purpose. However, it is preferred to use a C. glutamicum lysine production strain such as for ATCC13032 lysC^{fbr} or other derivatives of ATCC13032 or ATCC13286. The detailed construction of ATCC13032 lysC^{fbr} is described in patent application WO2005059093.

3.4 Determination of Expression of Optimized lysA Gene and Lysine Production.

Once one will have obtained the C. glutamicum strain ATCC13032 lysC^{fbr} derived strains comprising the optimized lysA gene several of these clones can be selected to be investigated as to any effect on lysine productivity.

To analyze the effect of the codon usage optimized synthetic ddh or lysA genes on lysine productivity, the optimized strains are compared to lysine productivity of the parent strain.

To this effect one can grow the strains on CM-plates (10% sucrose, 10 g/l glucose, 2.5 g/l NaCl, 2 g/l urea, 10 g/l Bacto Pepton, 10 g/l yeast extract, 22 g/l agar) for two days at 30° C. Subsequently cells can be scraped from the plates and re-suspended in saline. For the main culture one can grow 10 ml of medium 1 and 0.5 g autoclave CaCo₃ in a 100 ml Erlenmeyer flask together with the cell suspension up to an OD₆₀₀ of 1.5. The cells are then grown for 48 hours on a shaker of the type Infers AJ118 (infers, Bottmingen, Switzerland) at 220 rpm. Medium 1 has the following concentration:

### Medium 1:

| | | |
|---|---|---|
| 40 | g/l | Sucrose |
| 60 | g/l | Melasse (calculated on the basis of 100% sugar content) |
| 10 | g/l | (NH₄)₂SO₄ |
| 0.4 | g/l | MgSO₄*7H₂O |
| 0.6 | g/l | KH₂PO₄ |
| 0.3 | mg/l | Thiamine*HCl |
| 1 | mg/l | Biotin |
| 2 | mg/l | FeSO₄ |
| 2 | mg/l | MnSO₄ |

adjusted to pH 7.8 with NH₄OH autoclaved (121° C., 20 min) additionally Vitamin B12 (Hydroxycobalamine, Sigma Chemicals) is added to a final concentration of 100 μg/l.

Subsequently, one can determine the concentration of lysine that is segregated into the medium. This can be achieved by determining the amino acid concentration using HPLC on an Agilent 1100 Series LC system HPLC. A precolumn derivatisation with ortho-phthalaldehyde allows to quantify the formed amino acid. The separation of the amino acid mixture can be done on a Hypersil AA-column (Agilent).

The effect of using the optimized synthetic gene for lysA on the protein amount can be determined using 2D PAGE. A method how to perform 2D PAGE with proteins of Corynebacterium glutamicum can be found e.g. in Hermann et al. (Electrophoresis (2001), 22, 1712-1723). For the 2D PAGE analysis preferably medium without complex carbon- and nitrogen-sources is used.

It is assumed that the strains containing the optimized gene for lysA will comprise higher amounts of LysA protein compared to the wild type or parent strains that use the endogenous lysA sequence.

One may also determine the positive influence of a synthetic codon usage optimized genes by measuring the activity of lysA. This may be done by the method described in White P. J. Methods in Enzymology, 1971, 17, 140-145

4. Improved Protein Expression of lysA in C. glutamicum

In the following it is described how to increase the amount of cob(I)alamin-dependent methionine synthase (metH) by adapting the codon usage as mentioned above.

The enzyme metH is important for methionine biosynthesis. The wild type sequence of C. glutamicum metH is given as SEX) ID No. 10. The codon usage of the coding sequence of metH (SEQ ID No. 10) was determined using the Cusp function of the EMBOSS software package. The gene metH was amplified by PGR

Then, the codons corresponding to amino acid positions 53, 121 and position 154 were altered from G residues to C residues using established mutagenesis methods (Quikchange kit, Stratagene La Jolla USA) resulting in altered codons which still coded for glycine amino acids in the final protein metH (SEQ ID No. 11). The gene sequences of metH and the metH mutated amplified by PCR and fused with the sequence of the P_{GroES} promoter as described in WO 2005059143. The resulting genes were then cloned into the vector pCLIK5 MCS yielding the vector pCLIK5 MCS PGroES metH. In this vector the metH unmutated or metH mutated are transcribed under the control of the GroES promotor and are therefore expressed to significant levels in C. glutamicum as described in WO 2005059143. As a negative control, empty vector was used. In the case of the codon optimized metH gene the same vector was used as in the case of the normal form of metH

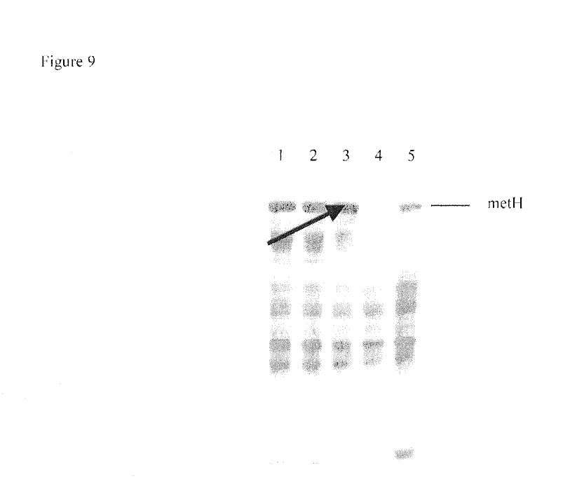

The genes were expressed In C. glutamicum as described in WO2007011845. It was found that strains expressing the mutated metH gene did show an improved and increased amount of metH protein as indicated by a gel band with increased staining and thickness (see FIG. 9).

1-28. (canceled)

29. A method of increasing the amount of at least one polypeptide in a host cell, comprising expressing in a host cell a nucleotide sequence encoding for at least one polypeptide, wherein the codon usage of the nucleotide sequence is adjusted to the codon usage of abundant proteins of said host cell, wherein the host cell is selected from microorganisms, insect cells, or plant cells.

30. The method of claim 29, comprising expressing in said host cell a modified nucleotide sequence encoding for said at least one polypeptide, wherein said modified nucleotide sequence is derived from a different starting nucleotide sequence and wherein the modified and starting nucleotide sequence encode for substantially the same amino acid sequence and/or function.

31. The method according to claim 30, wherein codons of the starting nucleotide sequence are exchanged in the modified sequence by more frequently used codons with codon frequency being based on codon usage of abundant proteins of the host cell.

32. The method of claim 31, wherein the modified nucleotide sequence uses for each replaced amino acid the most frequently used codons of the abundant proteins of the host cell.

33. The method of claim 29, wherein said at least one polypeptide is a polypeptide from an organism different than said host cell, an endogenous polypeptide of said host cell, or a mutated version thereof.

34. A method of increasing the amount of at least one polypeptide in a host cell, comprising expressing in Corynebacterium a modified nucleotide sequence coding for at least one polypeptide wherein said modified nucleotide sequence is derived from a starting nucleotide sequence such that the codon usage of the modified nucleotide sequence is adjusted to the codon usage of abundant proteins of Corynebacterium.

35. The method of claim 34, wherein the Corynebacterium is C. glutamicum.

36. The method according to claim 34, wherein at least one codon of the starling nucleotide sequence is replaced in the modified nucleotide sequence by one of the two most frequently used codons as set forth in Table 2.

37. The method of claim 34, wherein at least one, some or all codons of said modified nucleotide sequence for each amino acid are selected from the codon usage of Table 3.

38. A recombinant modified nucleotide sequence encoding for a polypeptide which allows for increased expression of said polypeptide in a host cell wherein the codon usage of the nucleotide sequence is adjusted to the codon usage of abundant proteins of said host cell.

39. The recombinant modified nucleotide sequence of claim 38, wherein at least one codon of a starting nucleotide sequence is exchanged in the resulting modified nucleotide sequence by a more frequently used codon with codon usage being based on the codon usage of abundant proteins of the host cell.

40. The recombinant nucleotide sequence of claim 39, wherein the modified nucleotide sequence uses for each replaced amino acid the most frequently used codon of the abundant proteins of said host cell.

41. The recombinant nucleotide sequence of claim 38, wherein the codon usage of the modified nucleotide sequence is adjusted to the codon usage of abundant proteins of Corynebacterium.

42. The recombinant nucleotide sequence of claim 41, wherein the Corynebacterium is C. glutamicum.

43. The recombinant nucleotide sequence of claim 41, wherein at least one codon of the starting nucleotide sequence is replaced in the modified nucleotide sequence by one of the two most frequently used codons as set forth in Table 2.

44. The recombinant nucleotide sequence of claim 41, wherein at least one, some or all codons of the modified nucleotide sequence are selected for each amino acid from the codon usage of Table 3.

45. A vector which is suitable for expression of a polypeptide in a host cell wherein the vector comprises the nucleotide sequence of claim 38.

46. A host cell comprising the nucleotide sequence of claim 38 or a vector comprising the nucleotide sequence.

47. A method for producing fine chemicals comprising utilizing the nucleotide sequence of claim 38, a vector comprising the nucleotide sequence, and/or a host cell comprising the nucleotide sequence or vector for producing fine chemicals.

48. The method of claim 46, wherein the fine chemicals comprise amino acids, sugars, lipids, oils, fatty acids, vitamins, lysine, cysteine, methionine, or threonine.


SEQUENCE LISTING (19 sequences)

SEQ ID NO: 1 - DNA, 1251 bp, Artificial Sequence
  Synthetic gene sequence of Lysine-2,3- aminomutase
atgatcaaccgccgctacgaactgttcaaggatgtgtccgatgcagattggaacgattggcgctggcaggtgcgcaaccgcatcgaaaccgtggaagaactgaagaagtacatcccactgaccaaggaagaagaagaaggcgtggcacagtgcgtgaagtccctgcgcatggcaatcaccccatactacctgtccctgatcgatccaaacgatccaaacgatccagtgcgcaagcaggcaatcccaaccgcactggaactgaacaaggcagcagcagatctggaagatccactgcacgaagataccgattccccagtgccaggcctgacccaccgctacccagatcgcgtgctgctgctgatcaccgatatgtgctccatgtactgccgccactgcacccgccgccgcttcgcaggccagtccgatgattccatgccaatggaacgcatcgataaggcaatcgattacatccgcaacaccccacaggtgcgcgatgtgctgctgtccggcggcgatgcactgctggtgtccgatgaaaccctggaatacatcatcgcaaagctgcgcgaaatcccacacgtggaaatcgtgcgcatcggctcccgcaccccagtggtgctgccacagcgcatcaccccagaactggtgaacatgctgaagaagtaccacccagtgtggctgaacacccacttcaaccacccaaacgaaatcaccgaagaatccacccgcgcatgccagctgctggcagatgcaggcgtgccactgggcaaccagtccgtgctgctgcgcggcgtgaacgattgcgtgcacgtgatgaaggaactggtgaacaagctggtgaagatccgcgtgcgcccatactacatctaccagtgcgatctgtccctgggcctggaacacttccgcaccccagtgtccaagggcatcgaaatcatcgaaggcctgcgcggccacacctccggctactgcgtgccaaccttcgtggtggatgcaccaggcggcggcggcaagaccccagtgatgccaaactacgtgatctcccagtcccacgataaggtgatcctgcgcaacttcgaaggcgtgatcaccacctactccgaaccaatcaactacaccccaggctgcaactgcgatgtgtgcaccggcaagaagaaggtgcacaaggtgggcgtggcaggcctgctgaacggcgaaggcatggcactggaaccagtgggcctggaacgcaacaagcgccacgtgcaggaataa

SEQ ID NO: 2 - DNA, 1521 bp, Artificial Sequence
  Vector Insert comprising gene sequence of synthetic Lysine-2,3-aminomutase pClik 5a MCS
atgactagttagctgccaattattccgggcttgtgacccgctacccgataaataggtcggctgaaaaatttcgttgcaatatcaacaaaaaggcctatcattgggaggtgtcgcaccaagtacttttgcgaagcgccatctgacggattttcaaaagatgtatatgctcggtgcggaaacctacgaaaggattttttacccatgatcaaccgccgctacgaactgttcaaggatgtgtccgatgcagattggaacgattggcgctggcaggtgcgcaaccgcatcgaaaccgtggaagaactgaagaagtacatcccactgaccaaggaagaagaagaaggcgtggcacagtgcgtgaagtccctgcgcatggcaatcaccccatactacctgtccctgatcgatccaaacgatccaaacgatccagtgcgcaagcaggcaatcccaaccgcactggaactgaacaaggcagcagcagatctggaagatccactgcacgaagataccgattccccagtgccaggcctgacccaccgctacccagatcgcgtgctgctgctgatcaccgatatgtgctccatgtactgccgccactgcacccgccgccgcttcgcaggccagtccgatgattccatgccaatggaacgcatcgataaggcaatcgattacatccgcaacaccccacaggtgcgcgatgtgctgctgtccggcggcgatgcactgctggtgtccgatgaaaccctggaatacatcatcgcaaagctgcgcgaaatcccacacgtggaaatcgtgcgcatcggctcccgcaccccagtggtgctgccacagcgcatcaccccagaactggtgaacatgctgaagaagtaccacccagtgtggctgaacacccacttcaaccacccaaacgaaatcaccgaagaatccacccgcgcatgccagctgctggcagatgcaggcgtgccactgggcaaccagtccgtgctgctgcgcggcgtgaacgattgcgtgcacgtgatgaaggaactggtgaacaagctggtgaagatccgcgtgcgcccatactacatctaccagtgcgatctgtccctgggcctggaacacttccgcaccccagtgtccaagggcatcgaaatcatcgaaggcctgcgcggccacacctccggctactgcgtgccaaccttcgtggtggatgcaccaggcggcggcggcaagaccccagtgatgccaaactacgtgatctcccagtcccacgataaggtgatcctgcgcaacttcgaaggcgtgatcaccacctactccgaaccaatcaactacaccccaggctgcaactgcgatgtgtgcaccggcaagaagaaggtgcacaaggtgggcgtggcaggcctgctgaacggcgaaggcatggcactggaaccagtgggcctggaacgcaacaagcgccacgtgcaggaataaagttctgtgaaaaacaccgtggggcagtttctgcttcgcggtgttttttatttgtggggcaactagttc

SEQ ID NO: 3 - DNA, 17 bp, Artificial Sequence
  Primer WKJ90
cctaacacagaaatgtc

SEQ ID NO: 4 - DNA, 20 bp, Artificial Sequence
  Primer WKJ65
cagtctgcatcgctaacatc

SEQ ID NO: 5 - DNA, 40 bp, Artificial Sequence
  Primer WKJ105
atcttcttggcagaactcatgggtaaaaaatcctttcgta

SEQ ID NO: 6 - DNA, 34 bp, Artificial Sequence
  Primer WKJ106
gagagagatctagatagctgccaattattccggg

SEQ ID NO: 7 - DNA, 6601 bp, Artificial Sequence
  Plasmid pClik 5a MCS Psod genom KamA
tcgatttaaatctcgagaggcctgacgtcgggcccggtaccacgcgtcatatgactagttagctgccaattattccgggcttgtgacccgctacccgataaataggtcggctgaaaaatttcgttgcaatatcaacaaaaaggcctatcattgggaggtgtcgcaccaagtacttttgcgaagcgccatctgacggattttcaaaagatgtatatgctcggtgcggaaacctacgaaaggattttttacccatgataaatagaagatatgaattatttaaagatgttagcgatgcagactggaatgactggagatggcaagtaagaaacagaatagaaactgttgaagaactaaagaaatacataccattaacaaaagaagaagaagaaggagtagctcaatgtgtaaaatcattaagaatggctattactccatattatctatcattaatcgatcctaacgatcctaatgatccagtaagaaaacaagctattccaacagcattagagcttaacaaagctgctgcagatcttgaagacccattacatgaagatacagattcaccagtacctggattaactcacagatatccagatagagtattattattaataactgatatgtgctcaatgtactgcagacactgtacaagaagaagatttgcaggacaaagcgatgactctatgccaatggaaagaatagataaagctatagattatatcagaaatactcctcaagttagagacgtattattatcaggtggagacgctcttttagtatctgatgaaacattagaatacatcatagctaaattaagagaaataccacacgttgaaatagtaagaataggttcaagaactccagttgttcttccacaaagaataactccagaacttgtaaatatgcttaaaaaatatcatccagtatggttaaacactcactttaaccatccaaatgaaataacagaagaatcaactagagcttgtcaattacttgctgacgcaggagtacctctaggaaaccaatcagttttattaagaggagttaacgattgcgtacacgtaatgaaagaattagttaacaaattagtaaaaataagagtaagaccttactacatctatcaatgtgacttatcattaggacttgagcacttcagaactccagtttctaaaggtatcgaaatcattgaaggattaagaggacatacttcaggatactgcgtaccaacattcgttgttgacgctccaggtggtggtggaaaaacaccagttatgccaaactacgttatttcacaaagtcatgacaaagtaatattaagaaactttgaaggtgttataacaacttattcagaaccaataaactatactccaggatgcaactgtgatgtttgcactggcaagaaaaaagttcataaggttggagttgctggattattaaacggagaaggaatggctctagaaccagtaggattagagagaaataagagacacgttcaagaataaagttctgtgaaaaacaccgtggggcagtttctgcttcgcggtgttttttatttgtggggcaactagttcggacctagggatatcgtcgacatcgatgctcttctgcgttaattaacaattgggatcctctagacccgggatttaaatcgctagcgggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccacgctagcggcgcgccggccggcccggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaaggccggccgcggccgcgcaaagtcccgcttcgtgaaaattttcgtgccgcgtgattttccgccaaaaactttaacgaacgttcgttataatggtgtcatgaccttcacgacgaagtactaaaattggcccgaatcatcagctatggatctctctgatgtcgcgctggagtccgacgcgctcgatgctgccgtcgatttaaaaacggtgatcggatttttccgagctctcgatacgacggacgcgccagcatcacgagactgggccagtgccgcgagcgacctagaaactctcgtggcggatcttgaggagctggctgacgagctgcgtgctcggccagcgccaggaggacgcacagtagtggaggatgcaatcagttgcgcctactgcggtggcctgattcctccccggcctgacccgcgaggacggcgcgcaaaatattgctcagatgcgtgtcgtgccgcagccagccgcgagcgcgccaacaaacgccacgccgaggagctggaggcggctaggtcgcaaatggcgctggaagtgcgtcccccgagcgaaattttggccatggtcgtcacagagctggaagcggcagcgagaattatcgcgatcgtggcggtgcccgcaggcatgacaaacatcgtaaatgccgcgtttcgtgtgccgtggccgcccaggacgtgtcagcgccgccaccacctgcaccgaatcggcagcagcgtcgcgcgtcgaaaaagcgcacaggcggcaagaagcgataagctgcacgaatacctgaaaaatgttgaacgccccgtgagcggtaactcacagggcgtcggctaacccccagtccaaacctgggagaaagcgctcaaaaatgactctagcggattcacgagacattgacacaccggcctggaaattttccgctgatctgttcgacacccatcccgagctcgcgctgcgatcacgtggctggacgagcgaagaccgccgcgaattcctcgctcacctgggcagagaaaatttccagggcagcaagacccgcgacttcgccagcgcttggatcaaagacccggacacggagaaacacagccgaagttataccgagttggttcaaaatcgcttgcccggtgccagtatgttgctctgacgcacgcgcagcacgcagccgtgcttgtcctggacattgatgtgccgagccaccaggccggcgggaaaatcgagcacgtaaaccccgaggtctacgcgattttggagcgctgggcacgcctggaaaaagcgccagcttggatcggcgtgaatccactgagcgggaaatgccagctcatctggctcattgatccggtgtatgccgcagcaggcatgagcagcccgaatatgcgcctgctggctgcaacgaccgaggaaatgacccgcgttttcggcgctgaccaggctttttcacataggctgagccgtggccactgcactctccgacgatcccagccgtaccgctggcatgcccagcacaatcgcgtggatcgcctagctgatcttatggaggttgctcgcatgatctcaggcacagaaaaacctaaaaaacgctatgagcaggagttttctagcggacgggcacgtatcgaagcggcaagaaaagccactgcggaagcaaaagcacttgccacgcttgaagcaagcctgccgagcgccgctgaagcgtctggagagctgatcgacggcgtccgtgtcctctggactgctccagggcgtgccgcccgtgatgagacggcttttcgccacgctttgactgtgggataccagttaaaagcggctggtgagcgcctaaaagacaccaagggtcatcgagcctacgagcgtgcctacaccgtcgctcaggcggtcggaggaggccgtgagcctgatctgccgccggactgtgaccgccagacggattggccgcgacgtgtgcgcggctacgtcgctaaaggccagccagtcgtccctgctcgtcagacagagacgcagagccagccgaggcgaaaagctctggccactatgggaagacgtggcggtaaaaaggccgcagaacgctggaaagacccaaacagtgagtacgcccgagcacagcgagaaaaactagctaagtccagtcaacgacaagctaggaaagctaaaggaaatcgcttgaccattgcaggttggtttatgactgttgagggagagactggctcgtggccgacaatcaatgaagctatgtctgaatttagcgtgtcacgtcagaccgtgaatagagcacttaaggtctgcgggcattgaacttccacgaggacgccgaaagcttcccagtaaatgtgccatctcgtaggcagaaaacggttcccccgtagggtctctctcttggcctcctttctaggtcgggctgattgctcttgaagctctctaggggggctcacaccataggcagataacgttccccaccggctcgcctcgtaagcgcacaaggactgctcccaaagatcttcaaagccactgccgcgactgccttcgcgaagccttgccccgcggaaatttcctccaccgagttcgtgcacacccctatgccaagcttctttcaccctaaattcgagagattggattcttaccgtggaaattcttcgcaaaaatcgtcccctgatcgcccttgcgacgttggcgtcggtgccgctggttgcgcttggcttgaccgacttgatcagcggccgc

SEQ ID NO: 8 - DNA, 1338 bp, Artificial Sequence
  synthetic lysA sequence
atggctaccgttgaaaacttcaacgaacttcccgcacacgtgtggccacgcaatgccgtgcgccaggaagatggcgtggtgaccgtggctggtgtgcctctgcctgatctggctgaagaatacggaaccccactgttcgtggtggacgaagacgatttccgttcccgctgccgcgacatggctaccgcattcggtggcccaggcaatgtgcactacgcatccaaggcattcctgaccaagaccatcgcacgctgggtggatgaagaaggcctggcactggatatcgcatccatcaacgaactgggcatcgcactggcagcaggcttcccagcatcccgcatcaccgcacacggcaacaacaagggcgtggaattcctgcgcgcactggtgcagaacggcgtgggccacgtggtgctggattccgcacaggaactggaactgctggattacgtggcagcaggcgaaggcaagatccaggatgtgctgatccgcgtgaagccaggcatcgaagcacacacccacgaattcatcgcaacctcccacgaagatcagaagttcggcttctccctggcatccggctccgcattcgaagcagcaaaggcagcaaacaacgcagaaaacctgaacctggtgggcctgcactgccacgtgggctcccaggtgttcgatgcagaaggcttcaagctggcagcagaacgcgtgctgggcctgtactcccagatccactccgaactgggcgtggcactgccagaactggatctgggcggcggctacggcatcgcatacaccgcagcagaagaaccactgaacgtggcagaagtggcatccgatctgctgaccgcagtgggcaagatggcagcggaactaggcatcgatgcaccaaccgtgcttgttgagcccggccgcgctatcgcaggcccctccaccgtgaccatctacgaagtcggcaccaccaaagacgtccacgtagacgacgacaaaacccgccgttacatcgcagtggatggcggcatgtccgataacatccgcccagcactgtacggctccgaatacgacgcccgcgtagtatcccgcttcgcagaaggcgatccagtgtccacccgcatcgtgggctcccactgcgaatccggcgatatcctgatcaacgatgaaatctacccatccgatatcacctccggcgatttcctggcactggcagcaaccggcgcatactgctacgcaatgtcctcccgctacaacgcattcacccgcccagcagtggtgtccgtgcgcgcaggctcctcccgcctgatgctgcgccgcgaaaccctggatgatatcctgtccctggaagcataa

SEQ ID NO: 9 - DNA, 2560 bp, Artificial Sequence
  synthetic lysA sequence including up- and downstream regions
actatgacgtcggcgttgaagtcctgattggccagatggtgaacctgcttcgcgacggcaaggcagtgcgtatgtccaagcgtgcaggcaccgtggtcaccctagatgacctcgttgaagcaatcggcatcgatgcggcgcgttactccctgatccgttcctccgtggattcttccctggatatcgatctcggcctgtgggaatcccagtcctccgacaaccctgtgtactacgtgcagtacggacacgctcgtctgtgctccatcgcgcgcaaggcagagaccttgggtgtcaccgaggaaggcgcagacctatctctactgacccacgaccgcgaaggcgatctcatccgcacactcggagagttcccagcagtggtgaaggctgccgctgacctacgtgaaccacaccgcattgcccgctatgctgaggaattagctggaactttccaccgcttctacgattcctgccacatccttccaaaggttgatgaggatacggcaccaatccacacagcacgtctggcacttgcagcagcaacccgccagaccctcgctaacgccctgcacctggttggcgtttccgcaccggagaagatgtaacaatggctaccgttgaaaacttcaacgaacttcccgcacacgtgtggccacgcaatgccgtgcgccaggaagatggcgtggtgaccgtggctggtgtgcctctgcctgatctggctgaagaatacggaaccccactgttcgtggtggacgaagacgatttccgttcccgctgccgcgacatggctaccgcattcggtggcccaggcaatgtgcactacgcatccaaggcattcctgaccaagaccatcgcacgctgggtggatgaagaaggcctggcactggatatcgcatccatcaacgaactgggcatcgcactggcagcaggcttcccagcatcccgcatcaccgcacacggcaacaacaagggcgtggaattcctgcgcgcactggtgcagaacggcgtgggccacgtggtgctggattccgcacaggaactggaactgctggattacgtggcagcaggcgaaggcaagatccaggatgtgctgatccgcgtgaagccaggcatcgaagcacacacccacgaattcatcgcaacctcccacgaagatcagaagttcggcttctccctggcatccggctccgcattcgaagcagcaaaggcagcaaacaacgcagaaaacctgaacctggtgggcctgcactgccacgtgggctcccaggtgttcgatgcagaaggcttcaagctggcagcagaacgcgtgctgggcctgtactcccagatccactccgaactgggcgtggcactgccagaactggatctgggcggcggctacggcatcgcatacaccgcagcagaagaaccactgaacgtggcagaagtggcatccgatctgctgaccgcagtgggcaagatggcagcggaactaggcatcgatgcaccaaccgtgcttgttgagcccggccgcgctatcgcaggcccctccaccgtgaccatctacgaagtcggcaccaccaaagacgtccacgtagacgacgacaaaacccgccgttacatcgcagtggatggcggcatgtccgataacatccgcccagcactgtacggctccgaatacgacgcccgcgtagtatcccgcttcgcagaaggcgatccagtgtccacccgcatcgtgggctcccactgcgaatccggcgatatcctgatcaacgatgaaatctacccatccgatatcacctccggcgatttcctggcactggcagcaaccggcgcatactgctacgcaatgtcctcccgctacaacgcattcacccgcccagcagtggtgtccgtgcgcgcaggctcctcccgcctgatgctgcgccgcgaaaccctggatgatatcctgtccctggaagcataacgcttttcgacgcctgaccccgcccttcaccttcgccgtggagggcggttttgggtttctgggtttctttgcgaacttaaggtgtctaggggtctgcagtcgaggcttcagaggttttattgctcgacttcacgtgtgctgacttcgcgtgctcaacttctcatgaggcgacatcagtcgaaaagccggtcattgtcgtatgaagtctgcgcacgagaagttgagcggaagaagtcggtgttaagaagtcgaccgcgtgaagtcgccctttaggagaattctgactaactggagccaaaacttgatccactcgagagctgtgcagtctctttttccttcaattctgcctgctcgagctcgtagaagtagaggtctacttcagttggttcaccttgcacacaagcatgaagtagtgggtaggtcgagttgttaaatgcggtgtagaaggggagtagttcgctagcaaaggttaatttggagtcgctgtactgcgggttctcgggtggagtattcccggaggattcaagaaatcttgacgcatctttgatgaggtatgtttggaattcgtcggcaccttcctcgccggagaggtagtaggagttgtcgtctagaagtagt

SEQ ID NO: 10 - DNA, 3666 bp, Corynebacterium glutamicum
atgtcgacttcagttacttcaccagcccacaacaacgcacattcctccgaatttttggatgcgttggcaaaccatgtgttgatcggcgacggcgccatgggcacccagctccaaggctttgacctggacgtggaaaaggatttccttgatctggaggggtgtaatgagattctcaacgacacccgccctgatgtgttgaggcagattcaccgcgcctactttgaggcgggagctgacttggttgagaccaatacttttggttgcaacctgccgaacttggcggattatgacatcgctgatcgttgccgtgagcttgcctacaagggcactgcagtggctagggaagtggctgatgagatggggccgggccgaaacggcatgcggcgtttcgtggttggttccctgggacctggaacgaagcttccatcgctgggccatgcaccgtatgcagatttgcgtgggcactacaaggaagcagcgcttggcatcatcgacggtggtggcgatgcctttttgattgagactgctcaggacttgcttcaggtcaaggctgcggttcacggcgttcaagatgccatggctgaacttgatacattcttgcccattatttgccacgtcaccgtagagaccaccggcaccatgctcatgggttctgagatcggtgccgcgttgacagcgctgcagccactgggtatcgacatgattggtctgaactgcgccaccggcccagatgagatgagcgagcacctgcgttacctgtccaagcacgccgatattcctgtgtcggtgatgcctaacgcaggtcttcctgtcctgggtaaaaacggtgcagaatacccacttgaggctgaggatttggcgcaggcgctggctggattcgtctccgaatatggcctgtccatggtgggtggttgttgtggcaccacacctgagcacatccgtgcggtccgcgatgcggtggttggtgttccagagcaggaaacctccacactgaccaagatccctgcaggccctgttgagcaggcctcccgcgaggtggagaaagaggactccgtcgcgtcgctgtacacctcggtgccattgtcccaggaaaccggcatttccatgatcggtgagcgcaccaactccaacggttccaaggcattccgtgaggcaatgctgtctggcgattgggaaaagtgtgtggatattgccaagcagcaaacccgcgatggtgcacacatgctggatctttgtgtggattacgtgggacgagacggcaccgccgatatggcgaccttggcagcacttcttgctaccagctccactttgccaatcatgattgactccaccgagccagaggttattcgcacaggccttgagcacttgggtggacgaagcatcgttaactccgtcaactttgaagacggcgatggccctgagtcccgctaccagcgcatcatgaaactggtaaagcagcacggtgcggccgtggttgcgctgaccattgatgaggaaggccaggcacgtaccgctgagcacaaggtgcgcattgctaaacgactgattgacgatatcaccggcagctacggcctggatatcaaagacatcgttgtggactgcctgaccttcccgatctctactggccaggaagaaaccaggcgagatggcattgaaaccatcgaagccatccgcgagctgaagaagctctacccagaaatccacaccaccctgggtctgtccaatatttccttcggcctgaaccctgctgcacgccaggttcttaactctgtgttcctcaatgagtgcattgaggctggtctggactctgcgattgcgcacagctccaagattttgccgatgaaccgcattgatgatcgccagcgcgaagtggcgttggatatggtctatgatcgccgcaccgaggattacgatccgctgcaggaattcatgcagctgtttgagggcgtttctgctgccgatgccaaggatgctcgcgctgaacagctggccgctatgcctttgtttgagcgtttggcacagcgcatcatcgacggcgataagaatggccttgaggatgatctggaagcaggcatgaaggagaagtctcctattgcgatcatcaacgaggaccttctcaacggcatgaagaccgtgggtgagctgtttggttccggacagatgcagctgccattcgtgctgcaatcggcagaaaccatgaaaactgcggtggcctatttggaaccgttcatggaagaggaagcagaagctaccggatctgcgcaggcagagggcaagggcaaaatcgtcgtggccaccgtcaagggtgacgtgcacgatatcggcaagaacttggtggacatcattttgtccaacaacggttacgacgtggtgaacttgggcatcaagcagccactgtccgccatgttggaagcagcggaagaacacaaagcagacgtcatcggcatgtcgggacttcttgtgaagtccaccgtggtgatgaaggaaaaccttgaggagatgaacaacgccggcgcatccaattacccagtcattttgggtggcgctgcgctgacgcgtacctacgtggaaaacgatctcaacgaggtgtacaccggtgaggtgtactacgcccgtgatgctttcgagggcctgcgcctgatggatgaggtgatggcagaaaagcgtggtgaaggacttgatcccaactcaccagaagctattgagcaggcgaagaagaaggcggaacgtaaggctcgtaatgagcgttcccgcaagattgccgcggagcgtaaagctaatgcggctcccgtgattgttccggagcgttctgatgtctccaccgatactccaaccgcggcaccaccgttctggggaacccgcattgtcaagggtctgcccttggcggagttcttgggcaaccttgatgagcgcgccttgttcatggggcagtggggtctgaaatccacccgcggcaacgagggtccaagctatgaggatttggtggaaactgaaggccgaccacgcctgcgctactggctggatcgcctgaagtctgagggcattttggaccacgtggccttggtgtatggctacttcccagcggtcgcggaaggcgatgacgtggtgatcttggaatccccggatccacacgcagccgaacgcatgcgctttagcttcccacgccagcagcgcggcaggttcttgtgcatcgcggatttcattcgcccacgcgagcaagctgtcaaggacggccaagtggacgtcatgccattccagctggtcaccatgggtaatcctattgctgatttcgccaacgagttgttcgcagccaatgaataccgcgagtacttggaagttcacggcatcggcgtgcagctcaccgaagcattggccgagtactggcactcccgagtgcgcagcgaactcaagctgaacgacggtggatctgtcgctgattttgatccagaagacaagaccaagttcttcgacctggattaccgcggcgcccgcttctcctttggttacggttcttgccctgatctggaagaccgcgcaaagctggtggaattgctcgagccaggccgtatcggcgtggagttgtccgaggaactccagctgcacccagagcagtccacagacgcgtttgtgctctaccacccagaggcaaagtactttaacgtctaa

SEQ ID NO: 11 - DNA, 3666 bp, Artificial Sequence
  optimized Corynebacterium glutamicum metH
atgtcgacttcagttacttcaccagcccacaacaacgcacattcctccgaatttttggatgcgttggcaaaccatgtgttgatcggcgacggcgccatgggcacccagctccaaggctttgacctggacgtggaaaaggatttccttgatctggagggctgtaatgagattctcaacgacacccgccctgatgtgttgaggcagattcaccgcgcctactttgaggcgggagctgacttggttgagaccaatacttttggttgcaacctgccgaacttggcggattatgacatcgctgatcgttgccgtgagcttgcctacaagggcactgcagtggctagggaagtggctgatgagatgggcccgggccgaaacggcatgcggcgtttcgtggttggttccctgggacctggaacgaagcttccatcgctgggccatgcaccgtatgcagatttgcgtggccactacaaggaagcagcgcttggcatcatcgacggtggtggcgatgcctttttgattgagactgctcaggacttgcttcaggtcaaggctgcggttcacggcgttcaagatgccatggctgaacttgatacattcttgcccattatttgccacgtcaccgtagagaccaccggcaccatgctcatgggttctgagatcggtgccgcgttgacagcgctgcagccactgggtatcgacatgattggtctgaactgcgccaccggcccagatgagatgagcgagcacctgcgttacctgtccaagcacgccgatattcctgtgtcggtgatgcctaacgcaggtcttcctgtcctgggtaaaaacggtgcagaatacccacttgaggctgaggatttggcgcaggcgctggctggattcgtctccgaatatggcctgtccatggtgggtggttgttgtggcaccacacctgagcacatccgtgcggtccgcgatgcggtggttggtgttccagagcaggaaacctccacactgaccaagatccctgcaggccctgttgagcaggcctcccgcgaggtggagaaagaggactccgtcgcgtcgctgtacacctcggtgccattgtcccaggaaaccggcatttccatgatcggtgagcgcaccaactccaacggttccaaggcattccgtgaggcaatgctgtctggcgattgggaaaagtgtgtggatattgccaagcagcaaacccgcgatggtgcacacatgctggatctttgtgtggattacgtgggacgagacggcaccgccgatatggcgaccttggcagcacttcttgctaccagctccactttgccaatcatgattgactccaccgagccagaggttattcgcacaggccttgagcacttgggtggacgaagcatcgttaactccgtcaactttgaagacggcgatggccctgagtcccgctaccagcgcatcatgaaactggtaaagcagcacggtgcggccgtggttgcgctgaccattgatgaggaaggccaggcacgtaccgctgagcacaaggtgcgcattgctaaacgactgattgacgatatcaccggcagctacggcctggatatcaaagacatcgttgtggactgcctgaccttcccgatctctactggccaggaagaaaccaggcgagatggcattgaaaccatcgaagccatccgcgagctgaagaagctctacccagaaatccacaccaccctgggtctgtccaatatttccttcggcctgaaccctgctgcacgccaggttcttaactctgtgttcctcaatgagtgcattgaggctggtctggactctgcgattgcgcacagctccaagattttgccgatgaaccgcattgatgatcgccagcgcgaagtggcgttggatatggtctatgatcgccgcaccgaggattacgatccgctgcaggaattcatgcagctgtttgagggcgtttctgctgccgatgccaaggatgctcgcgctgaacagctggccgctatgcctttgtttgagcgtttggcacagcgcatcatcgacggcgataagaatggccttgaggatgatctggaagcaggcatgaaggagaagtctcctattgcgatcatcaacgaggaccttctcaacggcatgaagaccgtgggtgagctgtttggttccggacagatgcagctgccattcgtgctgcaatcggcagaaaccatgaaaactgcggtggcctatttggaaccgttcatggaagaggaagcagaagctaccggatctgcgcaggcagagggcaagggcaaaatcgtcgtggccaccgtcaagggtgacgtgcacgatatcggcaagaacttggtggacatcattttgtccaacaacggttacgacgtggtgaacttgggcatcaagcagccactgtccgccatgttggaagcagcggaagaacacaaagcagacgtcatcggcatgtcgggacttcttgtgaagtccaccgtggtgatgaaggaaaaccttgaggagatgaacaacgccggcgcatccaattacccagtcattttgggtggcgctgcgctgacgcgtacctacgtggaaaacgatctcaacgaggtgtacaccggtgaggtgtactacgcccgtgatgctttcgagggcctgcgcctgatggatgaggtgatggcagaaaagcgtggtgaaggacttgatcccaactcaccagaagctattgagcaggcgaagaagaaggcggaacgtaaggctcgtaatgagcgttcccgcaagattgccgcggagcgtaaagctaatgcggctcccgtgattgttccggagcgttctgatgtctccaccgatactccaaccgcggcaccaccgttctggggaacccgcattgtcaagggtctgcccttggcggagttcttgggcaaccttgatgagcgcgccttgttcatggggcagtggggtctgaaatccacccgcggcaacgagggtccaagctatgaggatttggtggaaactgaaggccgaccacgcctgcgctactggctggatcgcctgaagtctgagggcattttggaccacgtggccttggtgtatggctacttcccagcggtcgcggaaggcgatgacgtggtgatcttggaatccccggatccacacgcagccgaacgcatgcgctttagcttcccacgccagcagcgcggcaggttcttgtgcatcgcggatttcattcgcccacgcgagcaagctgtcaaggacggccaagtggacgtcatgccattccagctggtcaccatgggtaatcctattgctgatttcgccaacgagttgttcgcagccaatgaataccgcgagtacttggaagttcacggcatcggcgtgcagctcaccgaagcattggccgagtactggcactcccgagtgcgcagcgaactcaagctgaacgacggtggatctgtcgctgattttgatccagaagacaagaccaagttcttcgacctggattaccgcggcgcccgcttctcctttggttacggttcttgccctgatctggaagaccgcgcaaagctggtggaattgctcgagccaggccgtatcggcgtggagttgtccgaggaactccagctgcacccagagcagtccacagacgcgtttgtgctctaccacccagaggcaaagtactttaacgtctaa

SEQ ID NO: 12 - DNA, 31 bp, Artificial Sequence
  Primer Old 540
actatgacgtcggcgttgaagtcctgattgg

SEQ ID NO: 13 - DNA, 20 bp, Artificial Sequence
  Primer Old 541
tgttacatcttctccggtgc

SEQ ID NO: 14 - DNA, 43 bp, Artificial Sequence
  Primer Old 542
gcaccggagaagatgtaacaatggctaccgttgaaaacttcaa

SEQ ID NO: 15 - DNA, 39 bp, Artificial Sequence
  Primer Old 543
ggtcaggcgtcgaaaagcgttatgcttccagggacagga

SEQ ID NO: 16 - DNA, 19 bp, Artificial Sequence
  Primer Old 544
cgcttttcgacgcctgacc

SEQ ID NO: 17 - DNA, 34 bp, Artificial Sequence
  Primer Old 545
actacttctagacgacaactcctactacctctcc

SEQ ID NO: 18 - DNA, 19 bp, Artificial Sequence
  Primer Old 494
aaccgtggaaaacttcaac

SEQ ID NO: 19 - DNA, 19 bp, Artificial Sequence
  Primer Old 499
ttccagggacaggatatca